(19) United States
(12) Patent Application Publication
(10) Pub. No.: US 2019/0262212 A1
(43) Pub. Date: Aug. 29, 2019

(54) METHODS AND DEVICES FOR BIOFLUID FLOW ASSIST

(71) Applicant: James E. Schroeder, San Antonio, TX (US)

(72) Inventor: James E. Schroeder, San Antonio, TX (US)

(73) Assignee: THE BOARD OF REGENTS OF THE UNIVERSITY OF TEXAS SYSTEM, Austin, TX (US)

(21) Appl. No.: 16/286,963

(22) Filed: Feb. 27, 2019

Related U.S. Application Data
(60) Provisional application No. 62/635,915, filed on Feb. 27, 2018.

(51) Int. Cl. A61H 1/02

(52) U.S. Cl. CPC A61H 1/0296; A61H 2201/1207; A61M 2205/3303; A61H 2201/1609; A61H 2230/305; A61H 2201/1246; A61M 2205/3331

(57) ABSTRACT

Certain embodiments are directed to a biofluid flow assist device comprising (a) a collar configured to wrap at least partially around a subject's neck or limb; (b) at least one biofluid flow assist mechanism configured to be position over a target when the collar is positioned on the subject's neck or limb; and (c) a controller operatively connected to the biofluid flow assist mechanism. In certain aspects the biofluid is blood, lymph, and/or cerebrospinal fluid (CSF).

### CROSS REFERENCE TO RELATED APPLICATIONS

The present application claims priority to U.S. Provisional Application No. 62/635,915 filed Feb. 27, 2019, which is incorporated herein by reference in its entirety.

### STATEMENT REGARDING FEDERALLY FUNDED RESEARCH

None.

### BACKGROUND OF THE INVENTION

A. Field of the Invention

The invention generally concerns method and devices for providing biofluid flow assist to subjects in need thereof. In particular the methods and devices can be used to ameliorate neurocognitive degeneration (NCD) in subjects having abnormal blood supply or metabolic waste removal dynamics.

B. Description of Related Art

Neurocognitive degeneration (NCD) is not only personally and financially devastating for victims and their families, but it is extremely costly for society in general. NCD is associated with a variety of medical conditions, but most commonly those involving a progressive and irreversible deterioration in cognitive processing and resulting in a chronic condition often described/labeled as “dementia.” Dementia is an umbrella term comprising a number of neurological conditions characterized by degraded cognitive and motor behavior, often accompanied by emotional/mood and personality changes.

NCD can be classified as primary dementia, in which it cannot be attributed to any other condition, or as secondary dementia, in which it can be attributed to another condition such as a substantial head injury. Most forms of dementia are related to old age, but age is neither a necessary nor sufficient condition for developing dementia. The overall incidence is unknown, but the overall prevalence for all forms of dementia in the United States was estimated by Plassman et al. (2007) to be 13.93% of the population 71 years of age and older, and it includes anyone diagnosed with Alzheimer's disease (AD—69.9% of all cases, with hallmarks comprising the accumulation of amyloid beta (Aβ) plaque and the presence of neurofibrillary tangles), vascular dementia (VaD—17.4% of all cases—usually associated with cardiovascular conditions), and “other types” (12.7% of all cases, and which includes dementia with Lewy-bodies (DLB—characterized by structures containing alpha-synuclein protein called Lewy bodies found in the mid-brain and sometimes in the cortex), Parkinson's dementia (PDm), normal-pressure hydrocephalus, frontal lobe dementia, alcoholic dementia, and traumatic brain injury. Lewy bodies also are associated with Parkinson's disease (PD) and other disorders, and often are found in the brains of people with AD, suggesting overlap between AD and DLB. Indeed, several investigators (e.g., Klucken et al., 2003; Guo et al., 2013; Moussaud et al., 2014; Vranova et al., 2014) have pointed out neurochemical similarities/links between AD and DLB. In addition to the more classic dementia patients, there are a number of other medical conditions associated with NCD which are not classified as dementia but possibly could benefit from the devices/methods described herein.

There is increasing evidence that NCD is related to poor blood supply to the neurons in the brain or poor removal from waste materials from the brain. The latter can involve poor movement of blood in the veins from the brain, poor movement of lymph in the lymph vessels from the brain, poor movement of cerebrospinal fluid (CSF) in the central nervous system, inadequate transfer of molecules from the interstitial fluid (ISF) to CSF, and inappropriate arterial pulsation—both because it is helps assure an adequate blood supply to the brain, and also because it has been implicated as a source for powering the adequate movement of biofluids involved in waste removal processes (i.e., blood, ISF, CSF, and lymph).

Interestingly, there are several approaches for restoring the natural movement of CSF in the central nervous system, including procedures from osteopathy and chiropracty (e.g., craniosacral therapy, adjustment of the spine and cranium, for vertebral misalignment, neural stretching technique, sacro-occipital technique, network spinal analysis, CSF technique, effleurage techniques directed to the cervical and thoracic lymphatics, manipulation of the spine, sacrum and ribs, massage of the levator scapulae, paravertebral muscles, accessory respiratory muscles, rhomboids, and trapezii, and an osteopathic technique of fourth ventricle compression to stimulate the cranial rhythmic impulse, and the CSF technique—which includes different chiropractic low-pressure and massage techniques along the spinal cord and the cranial bones), and various other breathing/postural techniques (e.g., rebirthing, transformational breathwork, holotropic breathwork, yogic breathing techniques such as alternate nostril breathing, and various yoga movements). The present invention also has similarities to a current noninvasive procedure called Enhanced External Counterpulsation (EEC), which targets the lower body and sequentially inflates cuffs surrounding the calves, thighs, hips, and pelvis, and then releases all pressure just prior to an arterial pulse, with the similar intent of helping return blood from the lower extremities. EEC currently has been approved by the FDA for treatment of chronic or unstable angina and congestive heart failure, and a recent meta-analysis supported the efficacy of using EEC for treating chronic refractory angina (Zhang et al., 2015).

There remains a need for additional methods and devices to treat and/or ameliorate NCD.

### SUMMARY OF THE INVENTION

Embodiments of the current invention provide a solution to the NCD problems associated with various patient populations. In particular, certain embodiments are directed to devices designed to assist the vascular system. By way of example, the inventor has designed a device to assist the vascular system, which results in augmenting the supply of nutrients and/or the removal of waste products from the brain. Without wishing to be bound by theory, it is believed that the use of device results in prophylactic treatment or conditioning of a subject, and amelioration of dementia, NCD, and other pathologies.

In addition to people with dementia, the current devices might be relevant for people suffering from other neurocognitive-related conditions, some of which are described in examples of conditions described below (e.g., PD, multiple sclerosis, multiple system atrophy, depression, mild cognitive impairment, frontotemporal degeneration, Down's syndrome, transient global amnesia, neurocardiovascular instability, chronic fatigue syndrome, amyotrophic lateral sclerosis, chronic traumatic encephalopathy, patients with altered blood pulsatility due to malfunctioning real or artificial heart valves, bypass surgery, hemodialysis machines, and some traumatic brain injuries).

Certain embodiments are directed to a biofluid flow assist device comprising (a) a collar configured to wrap at least partially around a subject's neck or limb; (b) at least one biofluid flow assist mechanism configured to be position over a target when the collar is positioned on the subject's neck or limb; and (c) a controller operatively connected to the biofluid flow assist mechanism. In certain aspects the biofluid is blood, lymph, ISF, or CSF. The device can comprise a first and second biofluid flow assist mechanisms. The biofluid flow assist mechanism can be a mechanical, electrical, or hydraulic vascular assist mechanism. The device can further include at least 1, 2, 3, 4, 5 or more sensors. A sensor can be an auditory sensor, pressure sensor, or a combination thereof. The device can have at least a first sensor and a second sensor. The first sensor can be positioned proximal to the biofluid flow assist mechanism relative to the subjects body or heart, and the second sensor can be positioned distal to the biofluid flow assist mechanism relative to the subjects body or heart. In certain aspects the biofluid flow assist mechanism is a lymph assist mechanism, CSF assist mechanism, ISF assist mechanism, vascular assist mechanism, arterial assist mechanism, or venous vascular assist mechanism, or any combination thereof. In a particular aspect the device includes both an arterial assist mechanism and a venous assist mechanism. The target can be the common carotid artery, internal jugular vein, external jugular vein, vertebral artery, vertebral vein, myodural bridge, or combinations thereof. Other potential targets include vessels in the arm(s) (e.g., brachial artery, cephalic vein, basilic vein) and leg(s) (e.g., femoral artery, popliteal artery, tibial artery, femoral vein, popliteal vein, saphenous vein). As an example, for individuals determined to have deficient cerebral pulse pressure (PP) and also unable to wear a neck collar, applying moderate pressure to the brachial artery during systole could increase the systolic pressure being delivered to the brain (i.e., increase PP). In addition, the device controller can be programmed to control one or more biofluid flow assist mechanisms and the synchrony among multiple biofluid flow assist mechanisms; in addition, each biofluid flow assist mechanism can be programmed with regard to onset, frequency, duration, force, and force pattern.

Other embodiments are directed to methods of assisting biofluid movement in a subject, e.g., in the brain or cardiovascular system, comprising positioning a biofluid flow assist device on a subject and in an appropriate location (e.g., neck, arm, leg, etc.), wherein the biofluid flow assist device is programmed to apply an appropriate series of pulses to a target for an appropriate duration. In certain aspects the method include configuring or controlling the device to deliver pulsed positive, negative, or positive and negative pressure of various durations. In certain aspects the pressure can be at most, at least, or about 0.1, 0.5, 1.0, 5.0, 10, 15, to 20 kPa, including all values and ranges there between.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “ameliorating,” “inhibiting,” “reducing,” or any variation of these terms includes any measurable decrease or inhibition to achieve a desired result.

The term “effective,” as that term is used in the specification and/or claims, means adequate to accomplish a desired, expected, or intended result.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B shows side views of two general styles for the device's collar; both are relatively wide. (FIG. 1A) Depicts a collar style that is open in the front, implying that this type is constructed with a material that is relatively rigid in order to stay in place and not absorb/diminish the forces to be applied to the neck. (FIG. 1B) Depicts another general style that is closed in the front; this design does not require the collar to be as rigid to stay in position.

FIGS. 2A-2B shows side views of a narrower collar with an open front that can be worn at different cervical levels as shown in (FIG. 2A) (relatively high placement) and (FIG. 2B) (relatively low placement); the actual height to be determined as indicated by the condition being treated, individual differences in the anatomy of the neck, and the location of the targeted artery or vein. The narrower collar also can be closed in the front (not depicted), but care must be taken to not place undue pressure on the larynx.

FIGS. 3A-3B presents side views for one general method of applying stimulation to both the carotid artery and jugular vein (FIG. 3A), or exclusively to either the carotid artery or jugular vein independently (FIG. 3B), by activating a compression mechanism (e.g., electrical, hydraulic, or hydraulic actuator) at the corresponding site(s) on one or both sides of the neck to apply positive or negative pressure on the skin (and tissue structures below the skin) at those sites. A power/control unit is programmed to activate the stimulation mechanism at a specific site based on a predetermined temporal distribution (i.e., onset, frequency, pattern, and duration are programmed). The associated power supply(ies) and control unit(s) can be contained inside an enlarged support collar (not shown) or, as shown in both frames, at another site (e.g., worn on the belt) and connected to the collar and stimulation mechanism(s) by a control cable or conduit (for pneumatic or hydraulic components). Also shown in (FIG. 3A) are arrays of auditory sensors on the proximal and/or distal sides of the stimulation mechanism (depicted on both sides); these optional auditory or pressure sensors are included to detect and relay normal pulsation information to the control unit, for example, in the event the patient's condition indicates that the applied pressure should be synchronous with the patient's natural blood pulse.

FIGS. 4A-4B presents essentially the same information as in FIGS. 3A-3B except that a narrow collar with an open front is depicted.

FIG. 5 shows a frontal view of a patient wearing a collar with an open front and an individual compression mechanism on both sides.

FIGS. 6A-6B presents transverse views of a collar with single compression mechanisms on each side; (FIG. 6A) shows a collar with a closed front and (FIG. 6B) shows a collar with an open front. Both frames depict one possible spatial relationship between the sites of compression and the corresponding targeted arteries/veins—exact locations depend on the targeted vessel(s), the corresponding cervical level, individual anatomical differences with regard to location of the vessel(s), and other relevant anatomical locations—e.g., the relative locations of the thyroid gland, trachea, jugular vein valve, carotid bifurcation, hyoid bone, larynx, vocal cords, laryngeal prominence.

FIGS. 7A-7B depicts side views of a second general approach for applying pressure to a general area (as shown in FIG. 7A) or targeted for more specific site(s), such as the carotid artery (a) and/or jugular vein (b) in FIG. 7B. In this approach, a site where pressure is to be applied is more static (e.g., a skin-facing protrusion on the inside of the collar), and the actual force is applied by an actuator located in the split-half controller in the rear of the collar (or in the front of the collar, or both—neither shown), which, when activated, squeezes the two halves of the “split-half” collar together, indirectly applying a force at the targeted site(s). In such embodiments, the collar can be open or closed in the front (back if the split-half controller is in the front), and the collar can be relatively narrow or wide; however, the collar must be fairly rigid in the region ranging from the pressure site(s) to the split-half controller in order to transfer the proper pressure to the site(s).

FIGS. 8A-8B presents illustrative examples utilizing one stimulation site (FIG. 8A) or two stimulation sites (FIG. 8B) for applications targeting the vertebral arteries and/or vertebral veins.

FIG. 9 shows an example where the stimulation site is not a vascular vessel, but the suboccipital triangle, an area reported to create CSF movement when pressure from adjacent muscles are applied. Pressure/vacuum can be applied to that site, or muscles in that region which have been shown to create CSF movement can be electrically stimulated (the rectus capitis posterior major, obliquus capitis superior, and obliquus capitis—see Zheng et al., 2014 and Xu et al., 2016.

### DETAILED DESCRIPTION OF THE INVENTION

During exercise, pulse pressure (PP) increases from around 30-40 mmHg to around 100 mmHg in healthy individuals as a result of stroke volume increasing and total peripheral resistance decreasing. The resulting increase in PP contributes to both the increased supply of nutrient-rich blood to the neurons and the removal of waste materials by facilitating the movement of ISF, CSF, lymph, and venous blood. However, for a variety of reasons, not everyone does or can exercise at a level that contributes to those increased activities. In addition, some people have a vascular system that is unable to provide an adequate blood supply to the neurons in the brain. Others have inadequate mechanisms for efficiently removing the resulting metabolic and other waste materials generated by normal neuronal metabolic activities. Aspects of the present invention have the capability of noninvasively augmenting pulsatility and shaping the pulse, including those delivered to the brain of an individual with inadequate pulsation who is not able to exercise or who has uncontrolled and elevated peripheral resistance. The present invention also has the potential to improve ISF/CSF material exchange, improve the movement of CSF to the veins, improve movement of lymph, and the improve movement of blood in the veins. It addresses these tasks by noninvasively applying stimulation (pressure, vacuum, or electrical stimulation) at specifically targeted sites.

In one general embodiment of the invention, pressure that is controlled for amplitude, duration, and timing (e.g., synchronized with the wearer's normal pulse), can be applied at specific targeted locations to create a pulse or augment, attenuate, or shape the form of a pulse sent through an artery into the brain. It has been shown in the literature that the application of external pressure can produce a well-defined “artificial pulse” (e.g., Aldrich et al. (2016) showed that the application of external pressure increased lymph movement in the legs of subjects during and after pneumatic compression therapy, and Maltz and Budinger (2005) used a linear actuator to apply momentary external forces to the brachial artery in the arm to generate a single controlled artificial pulse). In other patients, the normal movement of waste-bearing blood through the veins can be altered, resulting in venous reflux in some individuals (i.e., when their venous valves do not function properly and allow backflow), or resulting in restricted blood flow in some individuals (i.e., when their faulty venous valves block normal heartward blood movement). In certain aspects the assist mechanism applies a mechanical force to assist biofluid flow. This mechanical force can be an applied pressure (e.g., physical or hydraulic pressure, or pressure created by application of a vacuum). In another general embodiment of the present invention, pressure can be applied to selected sites to help restore proper venous blood flow in either of the above specified situations, for example, by applying an external pressure to the veins during the inter-pulse interval in the former case, or by applying a heartward moving external pressure to the vein(s) in the latter case. In some patients, the PP might be determined to be excessive and potentially harmful; the present invention can apply external pressure and/or vacuum to dampen and distribute the peak pressure of such pulses, providing an augmented windkessel effect. In some individuals, the arterial ingress problem or the venous egress problem might only exist on one side (e.g., only the right carotid artery is partially blocked). Unfortunately, there is no (known) laterality control mechanism with which, for example, the heart adaptively delivers one level of PP to the right hemisphere and a different level of PP to the left hemisphere—because PP emanates from a single source. In other general embodiments, the invention can be customized to apply appropriate pressure/vacuum to the arteries or veins on one side while doing nothing to the other side; or it can be customized to apply completely different strategies to the right and left vascular vessels. Finally, while some of the above conditions are mutually exclusive, more than one of the above strategies might be indicated in a single patient; different embodiments of the present invention can apply such multiple interventions.

In certain aspects mechanical pressure is applied by the inflation/deflation of a hydraulic cylinder or pneumatic bladder that applies a force to the subject's skin's surface. In other aspects a chamber can be formed between the assist device and the subject's skin, the chamber can be evacuated to apply a force by exposing a location to a lower air pressure.

Electrical assist mechanisms. In another general embodiment, instead of external pressure being increased or decreased to effect changes in the arterial or venous vessels, electrical stimulation is applied through one or more electrodes strategically positioned on a neck collar adjacent to targeted arteries, veins, or muscles. Variations of electrical nerve or muscle stimulation (ENS or EMS—also called neuromuscular electrical stimulation) can be used to evoke muscular actions in some biofluid vessels using existing technologies such as transcutaneous electrical nerve stimulation (TENS), or microcurrent electric neuromuscular stimulation (MENS) (see for example Hallen et al. [2010]). Alternatively, skeletal muscles can be the target because their contraction can exert indirect pressure on adjacent veins or arteries, or even increase CSF movement (as noted by Zheng et al., 2014 and Xu et al., 2016).

In certain aspects, a device can provide programmable alternatives for selectively applying positive or negative physical pressure or electrical stimulation at specifically identified sites; the magnitude, temporal pattern, duration, and onset/timing relative to the natural blood pulse also are programmable, with the overall profile designed to achieve a particular vascular-related outcome.

### Target Sites for Stimulation

Potential sites for stimulation include any locations on the neck or limbs at which the introduction of stimulation could affect the flow of biofluid in any artery, vein, lymph vessel, or CSF/ISF passageway. Examples of primary arterial target sites include the left and right common carotids, the left and right internal carotids, the left and right external carotids, left and right vertebral arteries, left and right deep cervical arteries, and any muscle triggering site on any muscle that is positioned contiguous to a targeted artery or which, for example, by applying external electrical stimulation to a site on that muscle contracts that muscle and in doing so, asserts pressure on a targeted artery. Examples of primary venous target sites include the left and right internal jugular veins, left and right external jugular veins, left and right vertebral veins (e.g., as they leave the foramen transversum between the sixth and seventh cervical vertebrae), left and right deep cervical veins. Other candidate sites include any muscle triggering site on any muscle that is positioned contiguous to a targeted vein, lymph vessel, or CSF passageway, or which, for example, by applying external electrical stimulation to a site on that muscle contracts that muscle and in doing so, asserts pressure on a targeted vein, lymph vessel, or CSF passageway. While the internal jugular veins and the vertebral veins are more obvious targets, the external jugular veins also are included as possible sites (e.g., at mid neck, where they cross the sternomastoid muscle—well above the two sets of valves, one usually located at the entrance to the subclavian vein and the second located about 4 cm above the clavicle), because in some individuals the occipital vein joins them instead of the internal jugular vein, and the occipital vein receives blood from both the superior sagittal sinus (through the parietal emissary vein) and the transverse sinus (through the mastoid emissary vein). Like the internal jugular veins, both the external jugular veins and the vertebral veins have valves which could become dysfunctional and interfere with the proper flow of blood from the brain. Although primarily supplying muscles, the deep cervical arteries are another potential site because of their connection with the occipital artery, which supplies blood to the dura mater in the posterior cranial fossa (which contains the brainstem and cerebellum). Other non-cervical sites in the arms and legs are included because some individuals might not be able to tolerate pressures to the neck and stimulation of such target sites might generate the intended effect (e.g., increased pulsatility) in some individuals (e.g., in the arm: brachial artery, cephalic vein, basilic vein, and in the leg: femoral artery, popliteal artery, tibial artery, femoral vein, popliteal vein, saphenous vein). As an example, applying moderate pressure to the brachial artery during systole could increase the systolic pressure being delivered to the brain in some individuals (i.e., increased cerebral PP at the expense of dampened PP in the arm).

Another primary site which is not vascular is the myodural bridge in the suboccipital triangle (at the posterior base of the skull). This site is included because Zheng et al. (2014) and Xu et al. (2016) reported an increase in CSF flow at the craniocervical junction when subjects rotated their head, and proposed that muscle action (especially the rectus capitis posterior major, obliquus capitis superior, and obliquus capitis muscles), acted as a pump that powered the observed CSF circulation. The myodural bridge is unique tissue that extends to the cervical spinal dura mater. At any of these sites, positive or negative physical pressure or electrical stimulation can be applied by this device for the purpose of increasing CSF flow. For example, pulsatile pressure can be applied directly to the myodural bridge in the suboccipital triangle or programmed electrical stimulation of the rectus capitis posterior major, obliquus capitis superior, or obliquus capitis muscles can help induce CSF circulation.

While there are a plurality of candidate sites available, those which are actually targeted in a clinical setting depend on the individual patient's situation. For simplification, in the following discussion and in most of the figures, the common carotid arteries and internal jugular veins will be used as example target sites; this approach is not intended to limit the invention to those sites. In addition, for the purpose of simplifying the descriptions, only one or two sites are specified in many of the examples and figures; in actual clinical settings, the number of sites addressed will vary depending on the patient's situation, could range from one to several, and could include arteries, veins, lymphatic pathways, or other critical sites such as the myodural bridge described above.

It is important to note that there are several health/safety-related issues associated with this device—see the below section dedicated to Health and Safety Risks and Precautions. In addition to a wide range of embodiments, some of which are described above, in the real world there are two general overriding representative embodiments: (1) the “diagnostic/fitting” general embodiment is more elaborate, with a plurality and variety of optional stimulation sites, a variety of stimulation sources, optional stimulation magnitudes, durations, patterns, etc. which are controlled/monitored by an external computer while medical imaging and instrumentation monitor the patient's arterial flow, venous flow, lymphatic flow, CSF movement, and other relevant variables—this form is used in a clinical setting to conduct preliminary analyses, testing, evaluation, and fitting; and (2) the “daily clinical use” general embodiment is a self-controlled, portable, and programmable (based on the prescribed pattern of stimulation at the different sites) device which is worn by the user during the day or while sleeping—as prescribed. With this distinction in mind, the following discussion addresses the features common to both general overriding embodiments.

### Fluid Flow Assist Device

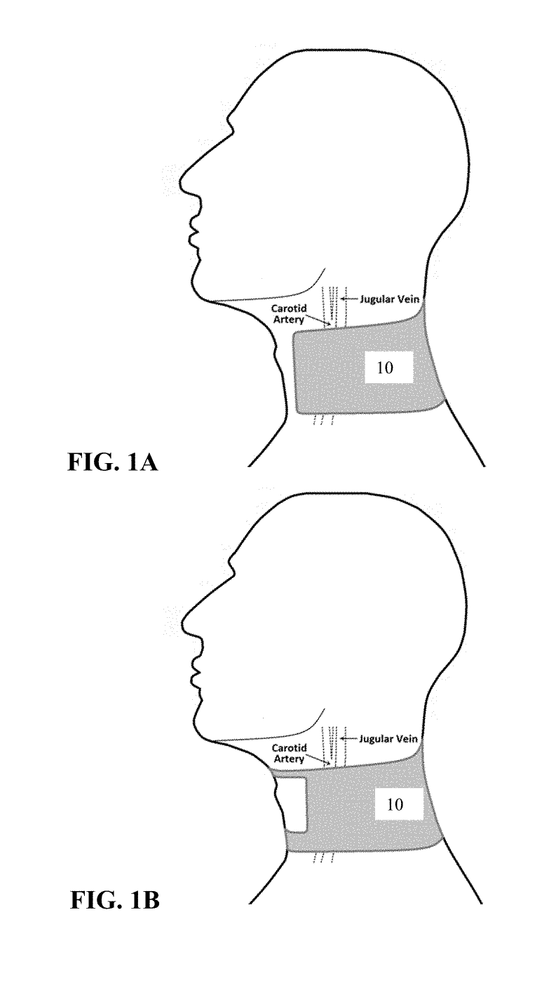

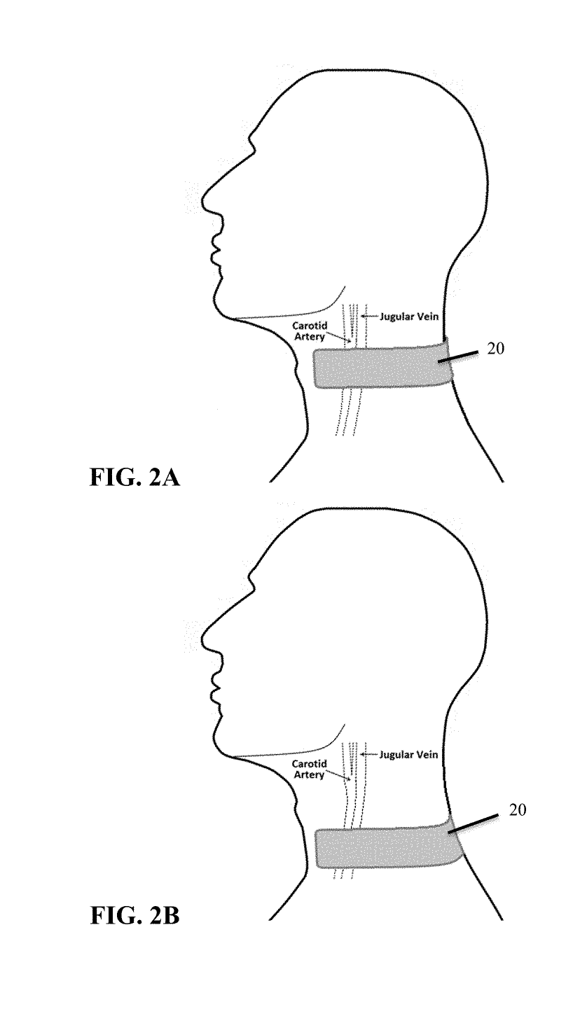

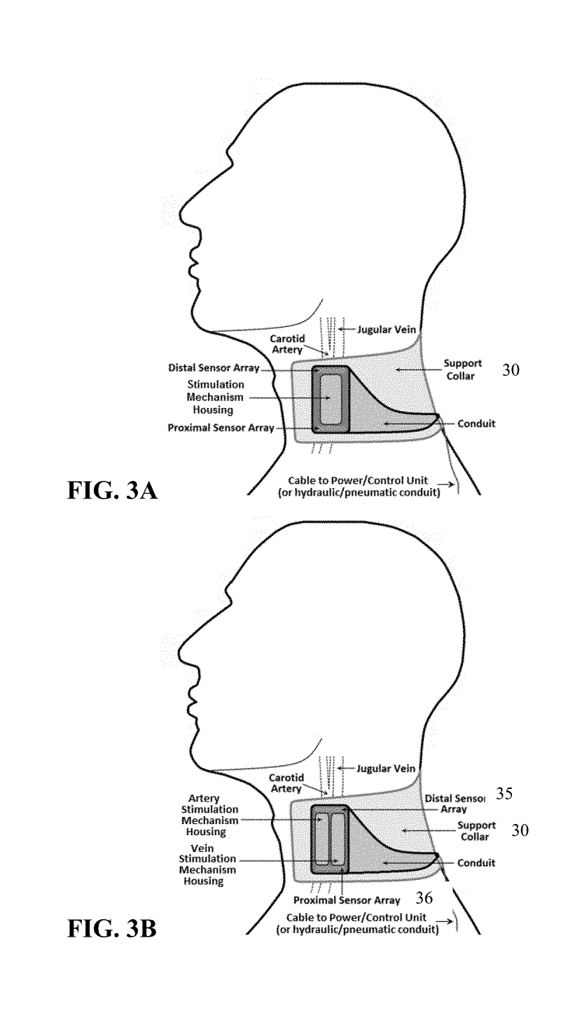

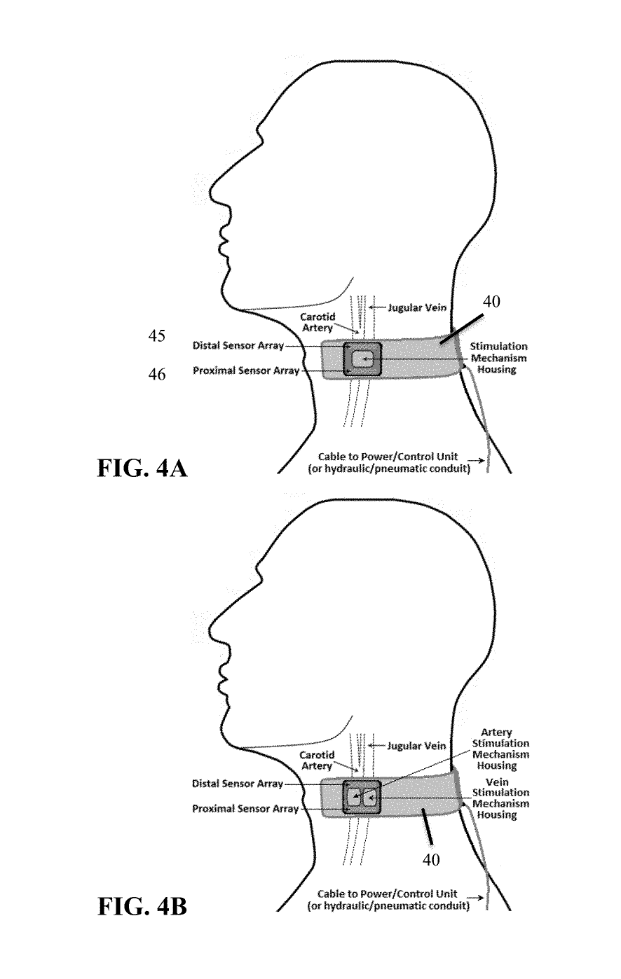

As shown in most of the figures, a neck apparatus (generally referred to as a “collar” below) is worn which serves as a platform for positioning stimulation mechanisms at targeted stimulation sites. Versions of the device can be designed for the limbs or other body locations. As shown in FIGS. 1A-1B, the neck collar 10 can be “split-half,” partially encircling the neck (FIG. 1A) or can completely encircle the neck (FIG. 1B). Either approach requires a joint or hinging mechanism which can be opened while donning/doffing the neck collar and closed and securely fastened once the collar is in place. In the case of the partial “split-half” enclosure (FIG. 1A), the walls of the platform are rigid and a clam-like hinge/joint provided at some point in the circumference (e.g., in the back), allowing easy on and off. In the case of a total enclosure (FIG. 1B), there is an opening in the front, allowing free movement of the laryngeal prominence, and the walls can be either rigid or relatively soft, flexible, or stretchable; however the region on the neck collar 10 that serves as the base for a mounted stimulation mechanism usually is harder, so that, for example, if positive pressure is being applied, it can be more carefully controlled, and to ensure that the pressure is being applied to the skin and not being absorbed by a soft platform. The neck collar can be relatively wide (e.g., FIGS. 1A-1B) or relatively narrow (e.g., FIGS. 2A-2B). The vertical placement of a narrow collar 20 can be relatively high (e.g., when the carotid artery is a target site—as shown in FIG. 2A), or relatively low (e.g., when the vertebral artery is a target site—as shown in FIG. 2B). The housing(s) for the stimulation mechanism(s) are mounted on the neck collar at site(s) appropriate for the patient's condition. A representative example of a single stimulation site (e.g., that exerts programmed rhythmic pressure on the common carotid artery or internal jugular valve, or both) is depicted in FIG. 3A for a wide neck collar 30 and FIG. 4A for a narrow neck collar 40. Representative examples of a dual stimulation site (e.g., that exerts independently programmed pressures on the common carotid and the internal jugular vein) are depicted in FIG. 3B for a wide neck band 30 and FIG. 4B for a narrow neck band 40.

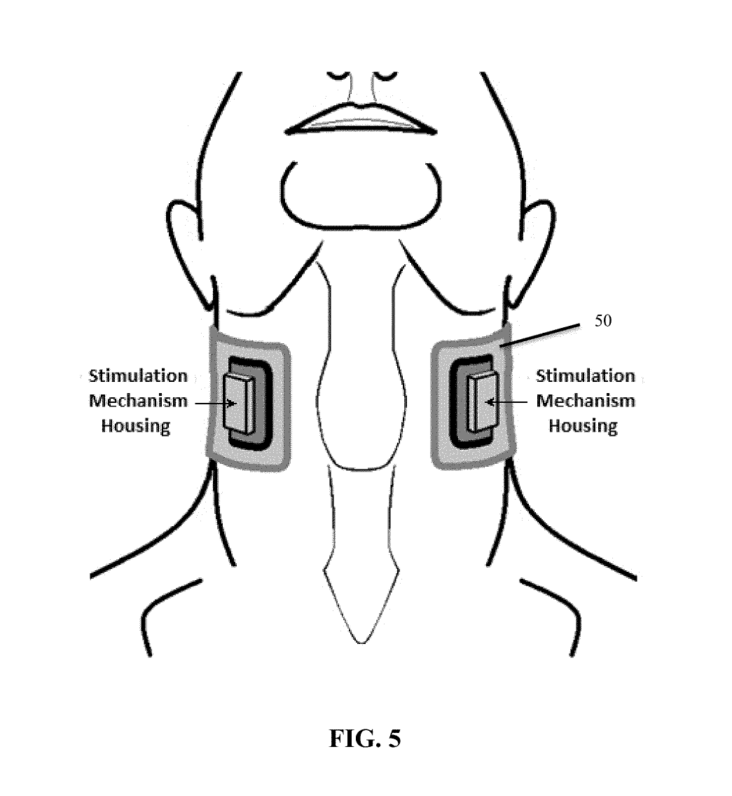

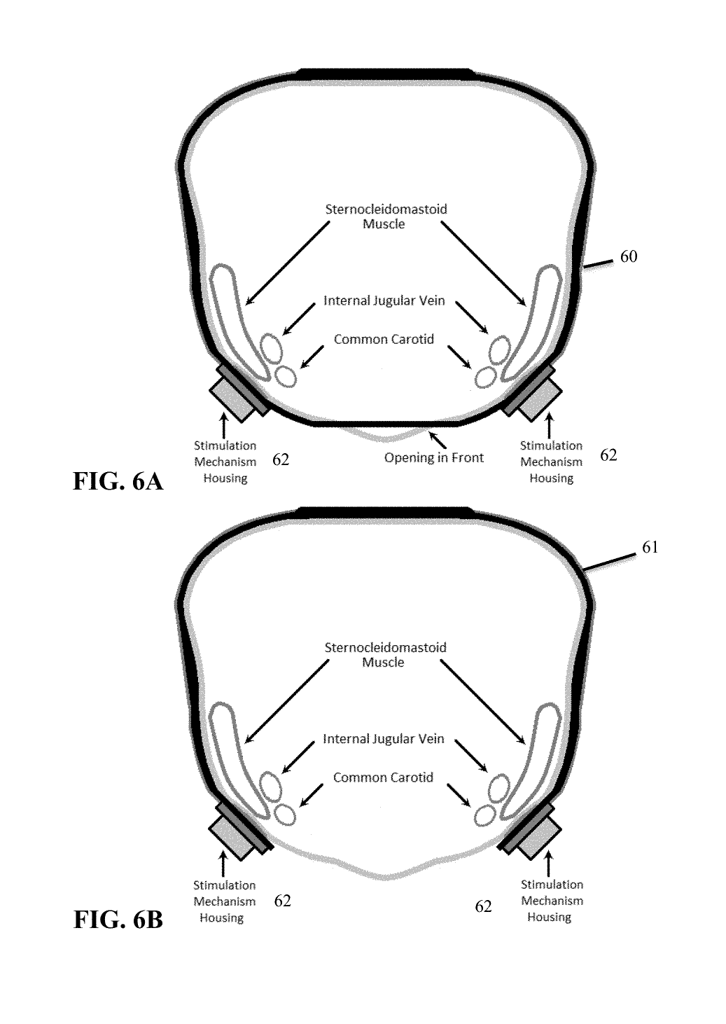

FIG. 5 presents a frontal view of a wide split-half neck collar 50 with one stimulation mechanism on each side. For some patient conditions, the stimulation mechanism might only be present or active on one of the two sides (not shown). FIGS. 6A-6B show a coronal “cross-sectional” views of a representative fully encircled neck collar 60 (FIG. 6A) and a split-half neck collar 61 (FIG. 6B). In both examples, the stimulation mechanism 62 is positioned so that it can apply external pressure simultaneously to both the common carotid artery and the internal jugular vein—believed to be a potentially useful embodiment for many patients because with it, pressure can be efficiently applied to both increase pulsatility and increase venous egress with each activation.

As described above, at any targeted stimulation site, the source of stimulation can be pressure or electrical stimulation. Pressure (positive or negative) can be generated using electrical devices (for example, solenoids or actuators or other electromagnetic mechanisms), hydraulically powered, or pneumatically powered. Appropriate voltage/current for electrical stimulation of muscles can be applied with properly positioned electrodes. Electrical, hydraulic, or pneumatic power sources can be contained within a compartment (e.g., a battery encased in the neck collar) and, or channeled via conduit or cable to a control unit mounted on the neck collar or to a more remote site—for example an appropriate electrical, hydraulic, pneumatic power source can be located in a separate unit worn on the belt by the user. Similarly, the programmable control unit can be mounted on or contained inside the collar, or reside in a separate control unit (e.g., worn on the belt of the user).

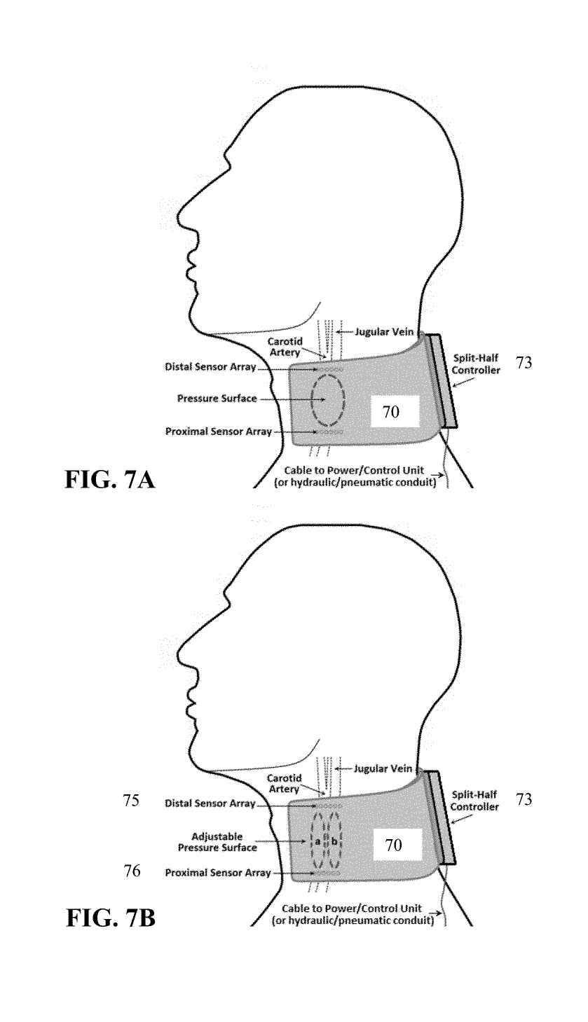

FIGS. 7A-7B depicts another embodiment appropriate for situations in which external positive pressure is to be applied at targeted sites. In this case, the stimulation sites are passive (e.g., simple protrusions projecting from the neck collar 70 toward the neck), and movement is generated in a split-half controller 73, located at the back hinged seam of the two halves. When activated, the split-half controller exerts pressure on the two halves, moving them toward each other, and hence, exerting pressure at the targeted stimulation site(s). FIG. 7A depicts an example in which pressure is exerted on both the common carotid artery and the internal jugular vein; FIG. 7B depicts another example in which pressure is more specifically targeted for one vessel or the other (i.e., the adjustable pressure site “a” is the only pressure site present if the carotid artery is the target and pressure site “b” is only present if the internal jugular vein is the target).

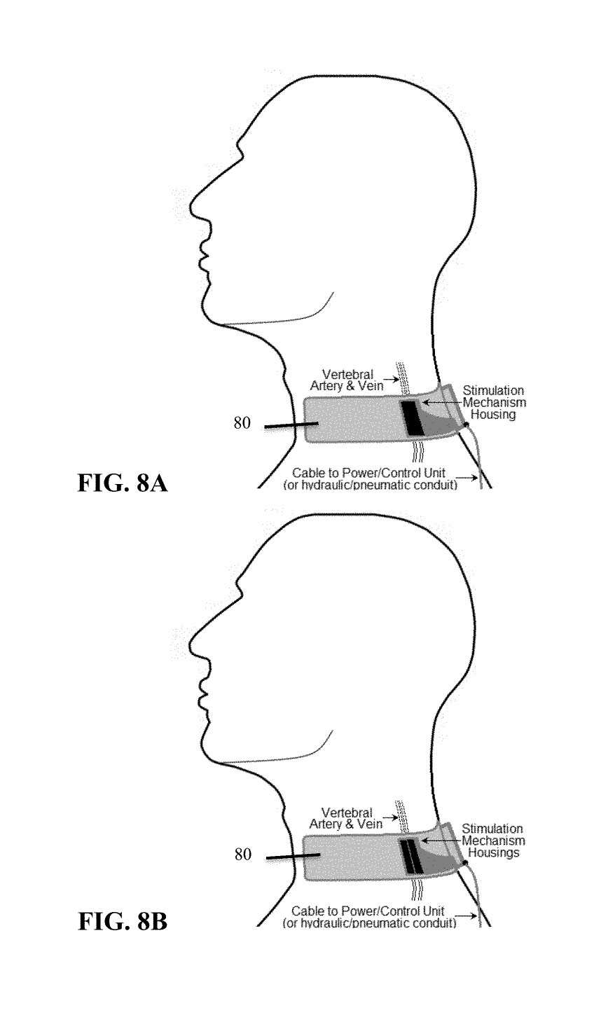

Other representative candidate target sites include the vertebral arteries and vertebral veins. While the exact locations of these targets are determined by imaging techniques, FIGS. 8A-8B show examples of one stimulation site (FIG. 8A) or two stimulation sites (FIG. 8B) at the approximate target sites for the vertebral artery and vertebral vein—where they emerge from the foramen transversum between the sixth and seventh cervical vertebrae.

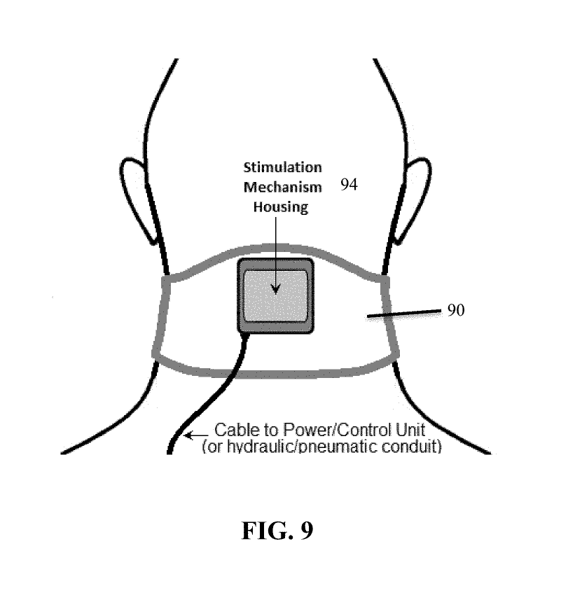

FIG. 9 depicts the myodural bridge stimulation site at the posterior base of the skull, which was suggested in the research findings of Zheng et al. (2014) and Xu et al. (2016). At this site, positive pressure, negative pressure, or both (e.g., in a repetitive alternating series) can be applied in order to increase CSF flow at the craniocervical junction. In other embodiments, electrical stimulation is applied to one or more of the three primary muscles in that region which were found to influence CSF flow: the rectus capitis posterior major, obliquus capitis superior, and obliquus capitis muscles.

Note that FIGS. 3A-3B, 4A-4B, and 7A-7B, show optional digital sensor arrays (35, 36, 45, 46, 75, 76), which each could include one or more pressure or auditory sensors (e.g., for auditory, microphones capable of detecting and relaying amplitude and frequency information about a passing arterial pulse and, to a lesser extent, passing venous blood movement). Any such sensor(s) are optional features for virtually any embodiment, and offer several possible enhancements. Because the spatial relationship between any given sensor and the stimulation site is constant and known, pressure/auditory information can be used to determine if the stimulation is being applied at the intended location. While a single sensor can be used for such estimates, there are potential advantages for using multiple sensors in a single array perpendicular to the targeted vessel(s)—as depicted in the figures. The relative magnitudes of the sound of the passing pulse across the array can be assessed and, for example, stored for future improvements (e.g., at the next clinic visit the horizontal location of a stimulation site can be adjusted fore/aft, so that it is positioned more directly over the intended target site). The sound pattern associated with a passing pulse contains not only information that can be stored and passed on to an attending healthcare provider, but also can be used to help control the device. For example, if a user is exercising, and hence producing a natural increase in heart rate and PP, the device could detect such events and interrupt any additional external stimulation. Even a single sensor could provide that level of input; for cost effectiveness, arrays of multiple sensors probably are more relevant for the diagnostic/fitting general embodiment (e.g., for positioning the stimulation mechanisms over the target sites) than for the daily clinical use general embodiment. Also note that those figures depicting digital sensor arrays show two arrays—one more proximal to the heart and one more distal to the heart, with one set obtaining auditory measures before the stimulation mechanism and one after the stimulation mechanism. Before-after information about passing arterial or venous blood can be stored for later analysis or can be actively used by the device to assess effectiveness—for example, by determining (a) whether an artificially generated pulse was actually introduced, (b) whether an actual arterial pulse was really dampened (as intended), (c) the speed of a passing pulse, (d) whether an artificially generated pulse was appropriately synchronized with an actual pulse, etc. Again, a single sensor positioned before (proximal) and after (distal) the stimulation site can accomplish much of this.

As described herein, syncope due to orthostatic hypotension, and possible pursuant falls are a major health concern for many in the targeted populations. For such patients, the neck collar or control unit can include sensors which detect when the wearer is in the act of sitting up or standing, and take steps to attempt to retain adequate blood pressure in the brain by, for example, temporarily augmenting arterial pressure during systole (e.g., by applying positive pressure to increase the next systole), retaining arterial pressure during dystole (e.g., by applying sustained—possibly repeated—positive pressure on the artery until the wearer's postural movement is completed or until an imposed safety release period has expired), while simultaneously reducing venous outflow (e.g., by applying sustained pressure on a vein until the wearer's postural movement is completed or until an imposed safety release period has expired), Kitano et al. (1964) found a general increase in cranial blood volume and pressure following sudden neck compression created by applying pneumatic pressure to the entire neck to close off the veins.

In the second general representative “daily clinical use” embodiment—a portable unit is worn by a patient during daily activities as prescribed by a healthcare provider. The unit would have a portable power source appropriate for the stimulation being delivered for that user (e.g., electrical, hydraulic, or pneumatic power source), and in any case, an electrical power source for powering the internal controls. The internal controls in the control unit(s) include microcircuitry and memory as necessary for storing data and for programming the delivery schedule, stimulation sites, and the nature of the stimulation (type, magnitude, pattern, duration, temporal pattern, etc.), as prescribed for that user. In addition, the portable control unit has self-monitoring capabilities with corresponding enforced rules, for example, pausing all scheduled stimulation in the event that an excessive magnitude or duration is detected.

### Customization and Use of the Device

Due to the safety considerations associated with the use of different versions of this device, it is critical under certain circumstances that its prescription, customization, and use be conducted under the supervision of qualified medical personnel. In general, use of this device depends on the patient's general health situation, which is determined by a thorough medical examination including appropriate tests, analysis of history data, imaging, symptomatology, etc. If the initial evaluation suggests a patient is a promising candidate for this device, then more intensive patient screening is conducted to determine the optimal schedule, stimulation sites, and the nature of the stimulation to be applied, along with real-time verification of the intended effects. Screening includes using established methods of testing for different types of neurocardiovascular instability (e.g., carotid sinus syndrome, orthostatic hypotension, vasovagal syndrome—see Kenny et al., 2002). Measures of venous pressure are obtained for any targeted veins on each side of the neck. Venous pressure above any valve can be compared to venous pressure below that valve or with central venous pressure to determine if a specific vein should be a target for applying external stimulation (i.e., to help blood pass through a dysfunctional valve that is restricting flow, to block blood from flowing backward, or both). Measures of CSF pressure and movement are obtained. Importantly, imaging can be used to assess several of the risks associated with the use of the devices/methods described herein (see section on Health and Safety Risks and Precautions).

The patient's vascular system is assessed by conducting appropriate medical tests to assess the presence/extent of atherosclerosis and to determine estimates of blood flow in the various candidate arteries and veins. Imaging can be used to determine the relative locations of all candidate vessels and adjacent vessels that might be affected by stimulation. Imaging also is used to determine the precise location of any nearby structures which must be avoided (e.g., the carotid bifurcation, thyroid gland, trachea, hyoid bone, larynx, vocal cords, laryngeal prominence). In general, the target sites for the carotid artery and internal jugular vein will be below the carotid bifurcation (and baroreceptors located there), above the internal jugular vein valve (or, if absent, where it is found in most people), and laterally, between the anterior edge of the sternocleidomastoid muscle and the occipital triangle (so as to not place pressure on the thyroid gland, trachea, hyoid bone, larynx, vocal cords, or laryngeal prominence). Stimulation should not be applied at the site of a vein valve (e.g., the IJVV); according to Valecchi et al. (2010), the mean location of the IJVV for 240 healthy individuals was 28 mm above the jugulo-subclavian junction, and similarly, Harmon & Edwards (1987) reported the mean location for a sample of 100 subjects as 17 mm above the brachiocephalic vein. Other medical comorbidities also must be considered, for example, patients with significant pain or other conditions in the cervical region of the spine could be injured or reinjured by a device exerting pressure on the neck.

As experience with using this invention increases, it is anticipated that different general patient profiles and corresponding stimulation strategies will emerge. Following is set of hypothetical patient profiles and their corresponding stimulation strategies. Type 1 patients are characterized by low blood pressure, low PP, and hypoperfusion in the brain; for such patients, a candidate strategy would be to apply external positive pressure to the main arteries going to the brain, or more specifically, to those determined by testing to have abnormally low PP and flow rates. Application of such pressure could be pulsatile and timed to coincide with the patient's natural pulse in order to magnify systole and PP. Because weak pulsatility is believed to be associated with poor CSF movement and corresponding poor ISF/CSF exchange, periodic positive pressure or alternating positive and negative pressure also could be applied to the myodural bridge, or electrical stimulation of adjacent/overlying muscles. Hypotension and orthostatic hypotension frequently are experienced by PD and DLB patients, possibly indicating a weak arterial pulse needs augmentation, not only in order to provide more blood to the brain, but also in order to increase the pulsatility required to move blood in the brain's veins, lymph in the brain's lymphatic vessels, and increase ISF/CSF movement and exchange in the CNS. The motor symptoms and corresponding deposits of Lewy bodies in the brainstem could indicate that the vertebral artery is a likely target.

Type 2 patients are characterized by indications of poor venous blood flow from the brain (i.e., in the jugular, vertebral, or other cervical veins) as indicated by, for example, high pressure distal to the valve or enlarged/filled veins distal to the valve. Such patients with vein blockage or with malfunctioning valves such that there is inadequate removal of blood from the brain might benefit from periodic application of pressure (perpendicular to, or in a “heartward” stroking fashion) distal to the dysfunctional valve. Patients without jugular vein valves or with valves that are malfunctioning such that blood passes freely in both directions might benefit from having a external pressure applied to that vein (serving as an artificial valve), which is periodically released so that blood can pass freely at critical times (e.g., after a set time period or after sufficient pressure has accrued to a point that reflux is unlikely), but is closed at other times to prevent reflux. In either case, timing of the onset and duration of such stimulations can be synchronized with venous pulsation if detectable, or estimated based on arterial pulsation. There is some support in the literature for the possibility that patients with MS fall into this group because of the number of studies associating jugular vein issues and jugular vein valve issues with MS. The relationship reported in the literature is far from perfect, but that could be due to the fact that most of the studies investigated only the internal jugular veins and their valves, while in fact, the vertebral veins and their valves might be implicated (because MS is associated with motor impairment), instead of or in addition to the internal jugular veins and their valves.

In a third hypothetical type, hypoperfusion exists despite normal to high PP, and atherosclerosis is present reducing the windkessel effect. One strategy for such Type 3 patients is to apply positive pressure to a targeted artery or transition from negative to positive pressure during diastole—in an attempt to create an artificial windkessel effect and help maintain a constant and sustained blood supply to the neurons. If CSF movement is below normal, then pulses of positive pressure or alternating positive and negative pressure also might be applied to the myodural bridge, or electrical stimulation of the adjacent muscles.

While arterial-driven pulsation is believed to be necessary for CSF circulation and ISF-CSF material exchange, there are studies in the literature suggesting that excessive arterial PP might damage neurons located close to the regions where arteries enter the brain (e.g., Alsop et al., 2008). Consequently, a hypothetical Type 4 patient is characterized by excessive PP, usually accompanied by atherosclerosis or arteriosclerosis (low arterial compliance), and poor CSF movement. The excessive PP (and high systolic blood pressure) could partially be due to unsuccessful homeostatic attempts to increase inadequate CSF movement, ISF/CSF exchange, and waste removal, more than a direct attempt to increase blood supply to the neurons. When excessive arterial PP is accompanied by inadequate CSF movement, then negative pressure can be applied during systole followed by a reversal to positive pressure in an attempt to reduce the peak pressure and re-distribute it over time (also possibly acting as an artificial windkessel effect), and CSF movement increased by applying pulses of positive pressure or alternating positive and negative pressure to the myodural bridge, or electrical stimulation of the adjacent muscles.

Similarly, some researchers have suggest that excessive CSF pulsation (often accompanied by excessive PP) could be the cause of neuronal damage leading to NCD conditions (e.g., O'Connell, 1943; Bering, 1962; Guinane, 1977; Di Rocco et al., 1978; Bateman, 2002; Greitz, 2004). In this hypothetical Type 5 patient, as in the previous type, a presumed homeostatic mechanism has detected inadequate waste removal or inadequate nutrition/oxygen supply, and arterial pulsation was increased, but unlike the previous type, the reaction was successful—CSF movement was increased; so the question is—why does elevated PP continue? One explanation for this outcome is that the problem is at another site in the disposal route, possibly in the movement of blood in the veins (similar to the explanation of communicating hydrocephalus suggested by Greitz, 2004). Consequently, treatment for this group might include steps to reduce excessive cerebral arterial pulsation by applying a negative to positive pressure sequence during systole as described in the previous type, while poor blood movement in the veins is concurrently addressed by applying positive pressure (stroking heartward if possible), to the implicated vein(s), facilitating movement of blood out of the brain.

Type 6 patients are characterized by abnormal heart performance (e.g., variable pulse magnitude, variable inter-beat intervals, variable pressure profiles among pulses). In a study unrelated to the present invention, Cooper & Hainsworth (2001) showed that heart rate could be influenced by applying positive or negative pressures to the neck. They showed that with or without pressure applied to the lower extremities (to simulate altered peripheral resistance), positive pressure applied to the neck lowered the pulse interval and negative pressure systematically increased the pulse interval. This outcome supports the notion that pressure applied to target arteries in the neck might be used to influence unusual heart performance—e.g., stabilize the pulse's magnitude, duration, temporal distribution, shape, frequency.

A seventh group of patients potentially benefiting from this approach is characterized by carotid sinus hypersensitivity, often accompanied by low blood pressure. Such Type 7 patients often are hypotensive and more prone to episodes of syncope—possibly because they have low blood pressure to begin with (i.e., their current average historical level is low, so it doesn't take as much change in blood pressure in the carotid sinus to elicit the carotid sinus reflex). If so, then any method applied that raises the average historical level could decrease the probability of premature elicitation of that reflex—because it would take additional external force to trigger the reflex. For such patients, the device initially could apply relatively low levels of external pressure, but over time, gradually increase the average blood pressure in the carotid sinus, presumably decreasing the probability that the protective reflex is elicited. Because these patients are susceptible to orthostatic hypotension and syncope, they also might benefit from steps targeted for that population as described in the next patient type.

Type 8 patients suffer from presyncope and syncope, (often accompanied by hypotension and hypoperfusion), and are likely to overlap the previous group. These symptoms are generally attributed to a redistribution of blood to the lower extremities when sitting up or standing up, resulting in reduced blood supply to the brain. While the carotid sinus reflex works to correct this, in some situations/individuals it cannot act quickly enough to sustain an adequate blood supply to the brain, resulting in presyncope, syncope, and falling. For such patients, the device could monitor arterial blood pressure in real time, sense sudden changes in orientation (standing or sitting up), and anticipatively apply pressure to the arteries to augment/sustain pressure in the carotid sinus and brain while also applying pressure to the cervical veins with the overall goal of sustaining blood pressure in the brain and thereby reducing the probability of presyncope, syncope, and falling.

A ninth group of patients who could benefit from this invention comprises patients with weak hearts or for whom the PP is weak because of inefficiency in their real or artificial heart valves. For such Type 9 patients, external positive pressure could be applied during systole, increasing the pressure at systole and the overall PP, presumably both increasing blood supply to the neurons and facilitating waste removal by improving ISF, CSF, and venous blood movement.

A tenth population includes patients undergoing kidney dialysis or heart bypass surgery. This group theoretically could benefit from receiving sustained normal arterial pulsatility during such procedures. While pulsation can be (perhaps should be) introduced or augmented within the apparatus of the external bypass machine, the proposed method offers the potential advantages of (a) fine tuning the PP magnitude, duration, and shape of the pulse entering the brain while the patient is on the machine to match those when the patient is off the machine (or to those for healthy age/sex-matched healthy individuals); and (b) the option of differentially “customizing” the incoming pulse in each individual artery, attempting to simulate the pulsatile pattern for each artery based on the individual's normal pattern or a standardized pattern based on healthy similar individuals.

While there are enough commonalities to support the possible existence of hypothetical clinical groups in order to relay the potential variations in the new approach, the literature suggests substantial overlapping symptomology among patients with different NCD-related diagnoses. The heterogeneity in the overall NCD population might be partially explained by underlying differences with regard to (a) the essential supply of blood to neurons and (b) the removal of the resulting waste materials. If there is an issue with a specific artery, lymph vessel, or vein, it could be limited to that vessel (and the region it services), or it could be due to a more general condition that affects other similar vessels (e.g., aging is related to thickening/hardening of the veins and lymph vessels as well as the arteries). The clinical approach proposed here is not to attempt to fit a new patient into such categories, but given an individual, (a) evaluate cardiac output (form, shape, distribution), (b) evaluate arterial function for all the implicated arteries independently (especially the presence of atherosclerosis), (c) evaluate venous function for all the implicated veins independently (especially, the presence of faulty valves and venous insufficiency), and (d) evaluate lymphatic flow and CSF movement; and then, given any abnormalities, attempt to normalize them by applying one or more of the noninvasive tools outlined above. Assessment of PP is believed to be extremely important in this approach; by applying specific stimulation at specific sites its magnitude, shape, and duration can be modified in a way that, in the long run, reduces NCD or the probability of its onset. Depending on the evaluation and resulting treatment goals, a user might wear this device most of the time, at times that are convenient for the user, or perhaps only at night while sleeping (e.g., Valdueza et al., 2000 showed that the jugular vein is more active when the body is in a supine orientation; Xie et al., 2013 reported that the exchange of CSF with extracellular fluid—an important step in the removal of waste—occurs during sleep; and Simmonds, 1952 and Lee et al., 2015a reported that in lower mammals, recline-related posture is associated with improved biofluid movement and CNS waste removal).

Health and Safety Risks and Precautions.

There are significant health risks associated with certain applications of this general method. For example, applying external pressure to an artery that has significant plaque buildup might cause the vessel wall to rupture or plaque attached to the wall of the artery to break loose, possibly triggering an embolism in the brain (ischemic stroke). If imaging determines significant levels of plaque in a patient's artery, then that patient might not be a suitable candidate for many of the methods described herein. As discussed above, if plaque is present, then further assessment can be made to estimate the risk of plaque rupture based on analysis of areas of high stress (Richardson et al., 1989; Versluis et al., 2006) using measures of plaque and vessel wall characteristics shown to be related to plaque rupture, as well as measures of strain gradient (Paini et al., 2007), and consideration of total cholesterol level, low HDL, and whether the patient is a male or smoker (Burke et al., 2002) or have type 2 diabetes or dyslipidemia (Paini et al., 2007)—all identified as risk factors for plaque rupture. In addition, risk can be reduced by applying a pretreatment (e.g., carotid endarterectomy used to remove the plaque), or by taking proper precautionary steps for at least the initial treatments (e.g., conducting initial tests in an appropriately equipped hospital/clinic, with appropriately trained medical personnel present, and countermeasures available, such as intravenous tissue plasminogen activator). In all cases, initial treatment applications must be conducted by properly trained medical personnel working in properly equipped medical facilities and using appropriate medical imaging and instrumentation to confirm that the desired effect is being produced by the device. In high-risk patients, all treatment might be confined to a clinic setting.

If external pressure is exerted on either of the carotid arteries, either directly on the carotid sinus or anywhere between the heart and the carotid sinus, then an important concern is its possible effect on the greater autonomic and cardiovascular system. As described above, the carotid sinus is the site of baroreceptors that monitor blood pressure in the carotid artery. If a sudden decrease in blood pressure is sensed by the baroreceptors, then an autonomic reflex action results in (among other things) an increase in cardiac output and an increase in peripheral resistance—with the net effect of providing more blood to the brain. Similarly, a sudden increase in blood pressure at those sites produces a very different outcome—decreased cardiac output (in extreme cases, cardiac arrest can occur), and reduced peripheral resistance. Patients with hypotension are more susceptible to presyncope and syncope. The rate of baroreceptor firing (which can trigger either outcome) is determined in part by the current blood pressure and the current PP relative to their average historical level. The average historical level is somewhat conditionable, so that for example, a person with hypertension has a higher average historical level than a person with “normal” blood pressure, who in turn has a higher average historical level than a person with hypotension—i.e., over time the average historical level is “conditioned” to match an individual's recent levels. Rate of baroreceptor firing is relative to the average historical level; so, especially in a person with hypotension, a sudden spurious increase in blood pressure can trigger a reduction in blood supply (decreased cardiac output and increased vascular resistance). Hence, it is possible that applying an external pressure pulse to the carotid artery as suggested in some variations of the present approach could trigger that outcome (i.e., vasodilated vessels, reduced cardiac output, presyncope, syncope, falling—even cardiac arrest in extreme cases). Consequently, it is critical that patients are screened for this possibility and that the any such proposed stimulation is programmable in a way such that, for example, initially, relatively small levels of additional pressure are applied, but over time, more pressure is applied in order to increase the average historical level to a more normal state. It is important that, at least initial and possibly all treatment sessions for hypotensive patients should be conducted in a highly controlled environment, with patients reclined and monitored closely by trained medical professionals.

Part of what is unknown is the effect of introducing an external pressure (introducing an artificial temporary increase in blood pressure) at one of the baroreceptor sites that results in input to the autonomic nervous system that is discrepant from information being received from the other baroreceptor sites in the body (e.g., the contralateral carotid sinus or aortic arch). However, adjustments can be made to the proposed method in attempt to counter risks/issues identified during clinical trials. For example, adjustments can be made to (1) the magnitude, duration, and “shape” of stimulations (i.e., the magnitudes of a stimulation across its duration), (2) the temporal relationship of each stimulation to the patient's actual pulse, and (3) the temporal distribution of stimulations, in attempt to minimize or maximize (depending on the patient's condition) the triggering of more systemic changes in blood pressure and cardiac output.

Despite the potential risks, for patients who are determined to be good candidates (e.g., have hypotension, hypoperfusion, and low PP), the proposed approach can produce three important outcomes: (1) increased blood perfusion and waste removal in the brain by gradually increasing PP; (2) reduced carotid sinus hypersensitivity (i.e., through extended use and conditioning, a non-invasively induced increase and stabilization of the blood supply to the brain and, by doing so, an increased historical pressure level in the carotid sinus and lower risk of eliciting an inappropriate autonomic/cardiovascular response); and (3) real-time detection of sudden changes in orientation (standing or sitting up) which triggers the application of pressure to the arteries (to augment/sustain pressure in the carotid sinus and the cervical veins, reducing the risks of syncope and falling).

In their study of AD patients, Alsop et al. (2008) noted that regions of increased blood flow in the brain coincided with the location of the major arteries and their first few branches. Increased flow in this region relative to healthy control subjects could be due to increased PP. The increased flow in the main arteries stood in remarkable contrast to the significantly reduced blood flow they found in the more peripheral cortical association areas—suggesting that a significant source of resistance was encountered downstream from the main arteries. Alsop et al. also noted that those arterial regions with increased flow corresponded to the physical locations at which Chetelat et al. (2002) had reported the earliest tissue loss occurring in AD patients. In a more recent related explanation, Wahlin et al. (2014) hypothesized that “excessive cerebral pulsatility damages the cerebral microcirculation, leading to brain atrophy in elderly persons.” In addition, O'Rourke & Hashimoto (2007) noted the potential harmful effects on microcirculation due to age-related pressures on downstream microvessels (due to stiffening of the aorta), and concluded: “The key to prevention and treatment is to minimize arterial pulsations, since these are the basic cause of large artery degeneration from elastin fiber fatigue and fracture, then of microvascular damage as pulsations are funneled into smaller vessels.”

It is important to note that strengthened PP created by a proposed method/device—at least in older patients and when the method involves applying external positive pressure to a targeted artery—could have the opposite effect of that intended, specifically, contributing to increased Aβ (Alsop et al.), or damaging microcirculation in the brain (Wahlin et al. and O'Rourke & Hashimoto). Consequently, augmenting PP with the an external device should be avoided or carefully considered for patients with already high levels of PP, or possibly applied to only those arteries where PP is not abnormally high. Alternatively, such a device might possibly be used to reduce the peak PP in such arteries as described herein.

Another possible adverse side effect of the proposed general approach is that if/when successful, significantly more waste material is transferred from the interstitial space in brain's parenchyma to the body's various disposal sites. This raises the possibility that the end-disposal organs could become overloaded with toxic material, suggesting that (a) use of this approach should be introduced slowly, with gradual increases over time; (b) the patient should be monitored carefully for such outcomes; and (c) preventative steps should be taken (e.g., modifying the treatment by reducing the duration or frequency of its operation, or by introducing drugs or other medical interventions to help the body deal with increased waste materials).

Finally, applying pressure to the skin and underlying muscles and blood/lymph vessels could result in tissue irritation or damage. Consequently, affected regions should be monitored by attending healthcare providers, and wearers should be instructed to be vigilant for any noticeable irritation or pain, and if present, discontinue use of the device, and report that outcome to their healthcare provider as soon as possible. To reduce the possibility of damaging blood vessels, it might be preferable in some individuals to apply pressure to adjacent muscles, cushioning the source of pressure while indirectly applying pressure to the target vessel. For example, pressure might be indirectly exerted on the internal jugular vein by applying external force to the sternomastoid muscle.

For the above identified risks and a variety of other reasons, it is critical that an extensive pre-assessment—such as that described above—be made of any patient determined to be a candidate for this method. If it then is determined that the treatment is potentially beneficial, all new patients must be adequately trained on using the device, and intensively monitored during its initial use. In addition, it is important that any such device be adjustable and programmable with regard to (1) the intensity of the external force applied, (2) the duration the external force is applied, (3) the location where the external force is applied, (3) the timing of the external force (e.g., the timing of the onset of any external force relative to the patient's natural pulse), and (5) the inter-stimulation temporal distribution of the applied force or current (e.g., in order to obtain the desired effect, there is no reason that distribution of artificial pulses must be identical to the patient's heart pulse, benefits might be realized if the external force is applied only once every 5 heart beats, every 10 beats, every 5 minutes, every half hour, etc.).

Conditions Associated with NCD

Commonalities Among the Conditions Associated with NCD. As described by Kotzbauer et al. (2001), there is considerable overlap in the symptoms among the different major types of dementia. Henry-Feugeas (2009) and others have noted that there are not clear-cut distinctions between AD and VaD, and that Lewy bodies (the hallmark for DLB) are found in other neurological disorders including PD, PDm, and AD.

The primary underlying factors for the following discussion and the empirical bases for the new family of devices and methods described herein are: (1) a primary cause for NCD, including not only the three major forms of dementia (AD, VaD, and DLB), but also a variety of other conditions associated with NCD as listed herein, results from the cumulative effects of inadequate blood supply to or inadequate removal of extracellular proteins and waste materials from the central nervous system (CNS), and (2) sub-optimal performance of the cardiovascular system is causally implicated in that dysfunction. Neither of these assertions is a new concept; they have been expressed by a variety of investigators working in a variety of areas as described below. What is new is the general approach for treatment and prevention made possible by the present invention. In the following sections, empirical evidence is presented which supports the assertion that there are common underlying contributing factors.

### Inadequate Waste Removal as a Contributing Factor to NCD

A number of researchers in the area of NCD have suggested that inadequate removal of extracellular proteins and metabolic waste material from the extracellular spaces in the brain parenchyma is a common factor in several NCD conditions, and most of those hypotheses were made in the context of reporting experimental results suggesting that possibility (e.g., Still, 1902; Weller et al., 1998; Shibata et al., 2000; DeMattos et al., 2002; Silverberg et al., 2003; Zamboni et al., 2009a,b; Weller et al., 2010; O'Leary et al., 2011; Hawkes et al., 2011; Nedergaard, 2013; Carare et al., 2013; Laman & Weller, 2013; Carare et al., 2014; Kida, 2014; Ethell, 2014; Kress et al., 2014; Simon & Illiff, 2016).

### Support for Cardiovascular Involvement as a Contributing Factor

Cardiovascular variables have been implicated in most forms of dementia, and the role of the cardiovascular system in causing or aggravating NCD is being increasingly recognized (e.g., Skoog, 1991; Henry-Feugeas, 2009; Gorelick et al., 2011). Several researchers have either suggested vascular issues as a common denominator (e.g., Neuropathology Group, 2001; Bateman, 2004; Bateman et al., 2005; Doepp et al., 2006; Purandare et al., 2006; Graban et al., 2009; Roberts et al., 2010; Kalaria et al., 2012; Wolf, 2012; Stellos et al., 2012; Ng et al., 2013; Carare et al., 2014; Kida, 2014; Ethell, 2014; Juurlink, 2015; Bateman et al., 2016), or reported no differences between AD and VaD patients in key cardiovascular measures (Morovic et al., 2009).

Poor cerebral blood flow has been reported in the frontal and parietal cortices in patients with subcortical ischemic vascular dementia (SIVD—a subtype of VaD, Schuff et al., 2009); and in the temporal and parietal cortices of patients with AD (Tohgi et al., 1998). Several investigators have reported cerebral hypoperfusion in patients with AD (e.g., Heun et al., 1994; Franceschi et al., 1995; Ruitenberg et al., 2005; de la Torre, 2009; Gorelick et al., 2011). Roher et al. (2012) reported that cerebral blood flow in patients with AD was 20% lower than for non-demented controls and the AD group had significantly lower pulse pressure (PP—one important measure of arterial stiffness that is measured by subtracting the diastolic blood pressure from the systolic blood pressure), and concluded: “A consensus is emerging that AD is a heterogeneous amalgam of multiple age-related neurodegenerative factors and vascular-associated pathologies.” Atherosclerosis in particular has been associated with dementia (e.g., van Oijen et al., 2007; Roher et al., 2011a).

Assuming that proper amounts of blood reach the neurons (which obviously remains as a necessary condition), the adequate removal of metabolic waste has now been recognized as a potentially critical component. The process is complex, involving the biochemistry and hydrodynamic physics of moving interstitial fluid (ISF), CSF, lymph, and eventually blood through the venous system to the body's various disposal sites.

### Overview of Waste Removal in the Central Nervous System

Providing oxygen- and nutrient-rich blood to the neurons in the CNS is an obvious and necessary requirement for sustaining neurocognitive health. Dysfunction in any one of the various systems involved in loading nutrients and oxygen into the blood is obviously a potential contributor to NCD, however, as a premise of the embodiments described herein it is assumed that nutrient- and oxygen-rich blood is being pumped from the heart and that waste materials after being transported to the end-disposal organs are being properly eliminated. Rather, the present discussion focuses on the fluid dynamics of moving blood from the heart to the neurons in the CNS (i.e., the output from the heart and movement of blood through the vessels transporting blood to the CNS via the arteries and arterioles in the cerebral arterial tree), and the subsequent fluid-based removal of waste materials via ISF, CSF, lymph and deoxygenated blood (called “nutrient-depleted blood” to distinguish it from “nutrient-rich blood” being pumped from the heart to the neurons), through the interstitial space surrounding the neurons, ISF/CSF passageways, lymph vessels, venules, and veins.

The removal of metabolic waste materials from the CNS involves a number of mechanisms and systems which are interrelated in complex ways that are just now starting to be understood. Great strides have been made in the past two decades, partly due to the availability of more sophisticated imaging tools. Below, a grossly simplified overview of normal blood supply and waste removal in the CNS is presented, followed by more detailed sections describing the primary systems involved.

Research indicates that certain arterial characteristics are required for the long-term health of the CNS. These requirements relate to the presence and specific physical characteristics of arterial pulsation—not only to provide blood to the neurons in the brain (i.e., blood could be provided without pulsation), but also because of the role pulsation has been shown to play in other CNS activities—including stirring/mixing/moving ISF and CSF to facilitate the exchange of waste materials from ISF to CSF, transferring waste from CSF into the veins for disposal, (likely role in) moving lymph through the recently discovered meningeal lymph vessels to the lymph nodes, and moving nutrient-depleted blood in the venous system back toward the heart.

After delivery from the arterioles to the neurons via ISF and CSF for metabolization, the resulting metabolic waste materials are transferred via the ISF and CSF in the interstitial space surrounding the neurons to the veins, where they eventually are transported to the heart via the jugular veins, the veins in the vertebral venous plexus (primarily the vertebral veins), the deep cervical veins, and other venous routes. In the following sections, the general path taken by the waste material is described in reverse order—i.e., starting with the venous system; the order is reversed in order to emphasize the importance of arterial pulsation in virtually every component. For each major component, known possible deviations/malfunctions which could interfere with proper disposal are identified along with associated NCD conditions.

### Venous System

Efficient removal of waste materials from the CNS via the veins is critical for neurocognitive health. In the veins, there is little/no remaining direct “in-line” pressure from the pulsing heart to push the nutrient-depleted blood back toward the heart, so other mechanisms are present to help provide the mechanisms necessary to move the blood heartward. In most of the body, external perpendicular pressures applied to the pliable walls of the veins by skeletal muscles and other externally and internally applied forces compress the veins and provide enough pressure to push the nutrient-depleted blood onward. Importantly, one-way valves in the veins prevent backflow (regurgitation), allowing nutrient-depleted blood to be pushed in a “lock-step” manner. Vein valves are especially important in the legs, where blood movement must overcome gravitational forces. It is commonly reported that there are no valves in any of the veins in the brain, so it has always been a mystery how blood is physically propelled in the brain. While it is true that eventually, gravity works to return blood from the head in an erect human, the superior sagittal sinus (one of the major venous sinuses in the brain) is located at the top of the brain, so gravity apparently is overcome even when in an upright posture. There must be other mechanisms in place which provide valve-like functions, allowing lock-step heartward movement of nutrient-depleted blood and preventing reflux.

A number of investigators have described how arterial pulsation periodically pressurizes the brain and contributes to venous outflow (e.g., Greitz et al., 1992; Greitz, 1993, 2004; Stivaros & Jackson, 2007)—not unlike the external perpendicular forces applied to the external walls of the veins by muscles in the rest of the body. The mechanisms they described can be interpreted as providing a valve-like mechanism. For example, in the very important subarachnoid space (SAS), periods of increased pressure resulting from the systolic phase of pulsation in the arteries located there is combined with the force of the brain parenchyma being enlarged and forced against the skull (e.g., Greitz, 2004; Bateman et al., 2005), resulting in overall increased pressure in that space and placing heightened external pressure on the pliable walls of the veins located there, momentarily closing them to blood flow. Such periods of vessel closure are followed by decreased pressure associated with the diastolic phase of pulsation in the SAS arteries and the movement of brain parenchyma away from the skull, producing a lower net pressure and allowing the elastic walls of the vein to dilate, opening the vessels for blood flow—the overall process is not unlike that of a valve powered by pulsation. In addition, arteries in the brain often are contiguous with veins (in parallel with veins or crossing over veins), and in the enclosed environment of the CNS, they are likely to directly or indirectly exert perpendicular pressure on the outside walls of the veins, perhaps providing the pressure that is typically applied by the skeletal muscles in the rest of the body. Pressure pulses in an artery that cross a vein might not only apply such external pressure, but also momentarily close the vein, providing a valve-like fashion if the walls of the artery and the vein are both compliant, and because the closure occurs at a point of initial arterial systole and terminal venous diastole, the closure might momentarily block blood from regurgitating while, by definition, initiating the next increase in upstream pressure that shortly thereafter indirectly powers the venous blood heartward.

Possible Sources of Venous Dysfunction.

The physical characteristics of the walls of the veins are critically important for their proper function. Just as aging affects the integrity of arteries (e.g., arteriosclerosis and atherosclerosis), aging can affect the integrity of vein walls. For example, phlebosclerosis, phlebothrombosis, and phlebitis all can produce structural changes in the veins and venules, possibly affecting their effectiveness or the effectiveness of their vein valves to function properly. Tzogias et al. (2011) concluded that, while controversy surrounds phlebosclerosis and more research is needed, that condition has been regarded as affecting the entire venous system—which includes the valves needed to direct/sustain blood flow. Just as arteries, veins must be “compliant” (i.e., elastic, flexible, pliable, soft—not hard or stiff), in order to function properly; contractions in the muscles of the vein's walls or perpendicular external pressures exerted on the vein's walls must compress them in order for the normal lock-step movement of nutrient-depleted blood to occur.

While the jugular veins typically are designated as the primary route of blood out of the brain, studies have shown that they are not always the primary venous drainage vessels, and their involvement can vary from individual to individual and even within individuals, for example, as a function of body orientation. Doepp et al., 2004 identified three types of people based on the percent of blood that flows through the jugular veins when in a supine body posture/orientation. The literature suggests that in healthy individuals, the primary path for blood flow out of the brain is through the internal jugular vein when subjects are in a supine orientation, but that flow is significantly altered when repositioned to a seated or upright orientation, with the internal jugular veins collapsing (e.g., Cirovic et al. 2003), and the drainage path from the brain rerouted to the vertebral venous system (e.g., Valdueza et al., 2000; Gisolf et al., 2004; Doepp et al., 2010). However the internal jugular vein can be reopened when a person is upright by increasing intrathoracic pressure (e.g., by performing the Valsalva maneuver—Gisolf et al., 2004).

A second general source of venous malfunction relates to the one-way valves involved in sustaining heartward movement and preventing blood reflux. Faulty vein valves can fail to prevent backflow, impede the heartward movement of blood, or both. Toro et al. (2015) noted both primary types of valve malfunction and reported that they were associated with venous reflux, intracranial hypertension, and redirection of blood flow. Valves that interfere with normal heartward movement of blood also can disrupt the normal pressure gradient, the change from relatively high pressure inside the brain to relatively low pressure in the superior sagittal sinus, to even lower pressure in the internal jugular vein. This pressure gradient is believed to be important for both blood removal and CSF circulation. As with the vein's walls, any vein valves that are too compliant could reduce their effectiveness in performing valvular functions (e.g., they might become easily inverted and allow reflux).

Neurological Disorders Associated with Venous Dysfunction.

One obvious condition that would impede the removal of waste materials is reduced flow of blood in the veins that drain the CNS—i.e., the internal jugular veins and other extra-jugular pathways including the vertebral venous system, intraspinal epidural veins, and deep cervical veins (see Schreiber et al., 2003). If such a condition persists, it is called chronic cerebrospinal venous insufficiency (CCSVI), and its association with NCD has been noted in the literature (e.g., Zamboni et al., 2009a, 2009c; Zivadinov & Chung, 2013; Mancini et al., 2014).

There are reports suggesting that decreased compliance in the cortical veins is causally related to hydrocephalus (Foltz & Aine, 1981; Foltz, 1984; Bateman, 2003), and studies have reported a significant relationship between CCSVI and multiple sclerosis (MS)—e.g., Zamboni et al., 2009b; Simka et al., 2010 However, other researchers have reported jugular vein associations with MS which were not consistent with the CCSVI hypothesis that venous congestion causes MS (e.g., Yamout et al., 2010; Baracchini et al., 2011; Zivadinov et al., 2011a), and Mayer et al. (2011) found no venous reflux and no difference in the cross sectional area of the jugular veins of MS patients compared to healthy controls. Despite the current lack of support for a direct single-causal relationship between CCSVI and MS, there are a number of studies reporting venous abnormalities in MS patients (e.g., Zaharchuk et al., 2011; Al-Omari & Rousan, 2010; Zivadinov et al., 2011b). According to Beggs et al. (2016), constricted internal jugular veins are associated with (a) increased blood volume in the brain (Kitano et al., 1964—although the procedure they used also compressed the carotid arteries); (b) increased intracranial pressure in a variety of patient populations—i.e., when pressure is exerted on the internal jugular vein by placing the patient in certain postures (Hulme & Cooper, 1976; Grady et al., 1986; Mavrocordatos et al., 2000; Ho et al., 2002); (c) stiffening of the parenchyma in the brain (Hatt et al., 2015); and (d) increased CSF pulsatility in the aqueduct of Sylvius (Beggs et al., 2014a; Hatt et al., 2015). In summary, while there appears to be evidence for jugular vein abnormalities in many MS patients, at this point it appears doubtful that CCSVI is the sole cause of MS. Potentially important for the present devices/methods, Schreiber et al. (2003) showed that when bilateral external pressure was placed on the jugular veins, blood flow in the vertebral veins significantly increased.

Another common condition affecting cerebral venous drainage is when the valves in the veins that drain blood from the CNS are absent or become damaged. Chronic venous insufficiency (CVI) is a condition that is related to malfunctioning or absent venous valves. In a review of the animal models in this area, Bergan et al. (2008) described the reflux of blood through incompetent vein valves as “a major cause of the venous hypertension that underlies clinical manifestations of chronic venous disease, including varicose veins, lipodermatosclerosis, and venous ulcers.”

Internal jugular vein valves can become incompetent, a condition called internal jugular vein valve incompetence (IJVVI), and which has been associated with NCD (e.g., Chung et al., 2014). There usually is one valve in each internal jugular vein and it is located just above the clavicle. Any reduction in the flow of blood in the venous system due to malfunctioning valves has implications for the effectiveness of the upstream mechanisms, and reduced CSF movement accompanying impaired cerebral outflow has been reported (e.g., Beggs et al., 2014a; Chung et al., 2014). A considerable body of literature has associated IJVVI with various NCD conditions—according to Simka et al. (2014), “ . . . incompetent jugular valves are prevalent in patients with transient global amnesia, transient monocular blindness, and Alzheimer's disease.” While IJVVI has been shown to be associated with a number of NCD conditions, it is unlikely to be the sole causal factor because, among other reasons, there is a fairly high incidence of incompetent or absent internal jugular vein valves even in healthy individuals (Valecchi et al., 2010). However, potential flaws in many of the above investigations of CCSVI and IJVVI are that (a) measurement/observation was limited to the internal jugular veins/valves—when it could be the other veins/valves involved in draining blood from the brain that are individually or collectively creating a drainage problem, and (b) body orientation is now known to affect which venous path(s) are involved, so the outcome of such studies could have been affected by the body orientation used during the study. In addition, while many studies have concentrated more on venous reflux, it is important to note that damaged vein valves also can produce the opposite effect—impeding blood flow and reducing the pressure gradient required for the drainage system to function properly; it also should be noted that research has concentrated on the possibility of defective venous valves affecting proper waste removal, but defective lymphatic valves also could be involved.

Transporting Waste Materials from the Interstitial Space to the Veins

In order for the venous system to relay waste materials to the final disposal sites in the body, waste must first be effectively collected by the ISF and CSF in the interstitial space surrounding the neurons and transported to and transferred to the veins. While the recently discovered lymphatic presence in the brain (Louveau et al., 2015; Aspelund et al., 2015; Absinta et al., 2017), is inevitably involved in this process, the exact mechanisms are not yet known. However, other functionally parallel mechanisms have been identified and are receiving considerable attention. For example, Nedergaard and his colleagues (e.g., Iliff et al., 2012) described how CSF enters the brain's parenchyma, interacts with ISF, and then transports the waste materials to the veins, labeling this process the “glymphatic system.” He later characterizing the glymphatic system as the “garbage truck of the brain” (Nedergaard, 2013).

One pathway for the evacuation of AP and other waste material from the brain is by drainage of ISF through perivascular pathways (e.g., Weller et al., 1998). Several investigators have proposed that arterial pulsation is critical for moving ISF and CSF, and that inadequate pulsation due to reduced compliance in aging arteries can reduce ISF movement, leading to inadequate waste removal and resulting in neurodegeneration (e.g., Schley et al., 2006; Carare et al., 2008; Hawkes et al., 2011; Wang & Olbricht, 2011; Iliff et al., 2012; Iliff et al. 2013a; Iliff et al. 2013b; Iliff & Nedergaard, 2013; Carare et al., 2013; Xie et al., 2013; Carare et al., 2014; Arbel-Ornath et al., 2013; Coloma et al., 2016). In a mathematical model, Schley et al. (2006) concluded “ . . . reduction in pulse amplitude, as in ageing cerebral vessels, would prolong the attachment time, encourage precipitation of Aβ peptides in vessel walls, and impair elimination of Aβ from the brain,” and that such events could contribute to NCD conditions in which excess Aβ accumulates in the brain such as AD. Similarly based on mathematical models, Wang & Olbricht concluded that “ . . . peristaltic motions of the blood vessel walls can facilitate fluid and solute transport in the PVS” (perivascular space) and Coloma et al. (2016) showed that reflected boundary waves from cardiac pulsations alone could create flow in the opposite direction (allowing the removal of waste materials), and that in an aging brain, the effects of basement membrane thickening (Farkas & Luiten, 2001), and stiffening artery walls (Nichols, 2005), “ . . . can lead to dramatic changes in transport direction and magnitude” (i.e., slowing the removal of waste material). Despite the increasing support for the notion that NCD is could be caused by deviations in underlying fluid dynamics, to date, biochemical solutions are the primary approaches being investigated and most serious investigators do not seem to even consider the possibility of more direct physical approaches when discussing therapeutic strategies.

The Role of Cerebrospinal Fluid.

Movement of CSF in the CNS has been described as the body's third circulatory system (Cushing, 1927). Inadequate CSF circulation has been hypothesized to cause poor health in general (Whedon & Glassey, 2009), and Rubenstein (1998) has proposed that changes due to aging (including cardiovascular changes, decreased compliance in brain tissue, and calcification of the choroid plexus—a network of blood vessels in each ventricle of the brain that produces CSF), can lead to reduced CSF circulation, which can contribute to some dementias.

Numerous investigators have suggested that movement of CSF and ISF is powered by arterial pulsation (e.g., Hadaczek et al., 2006; Schley et al., 2006; Hawkes et al., 2011; Arbel-Ornath et al., 2013; Kida, 2014). In healthy adults, Wahlin et al. (2014) reported a significant positive correlation between arterial and CSF flow volume pulsatilities. In addition, as described by Brinker et al. (2014), there is evidence that the perivascular space could serve as a drainage conduit for ISF into the lymphatic system, which is consistent with the fact that Aβ deposits have been found in the walls of arteries and arterioles of AD patients (Preston et al., 2003 and Weller et al., 2009). Besides blocking the flow of ISF and waste out of the brain, accumulation of Aβ in the perivascular space surrounding the arteries/arterioles could have the additional negative effect of attenuating the physical pulsatility of those arteries/arterioles, reducing the power source of the underlying pumping mechanism, or activating a homeostatic mechanism that increases arterial PP in order to sustain adequate PP at the perivascular level. In support of the critical role of arterial pulsation in CSF movement, studies have reported that brain movements (Feinberg & Mark, 1987; Levy et al., 1988; Greitz et al., 1992) and CSF movement from the cranial cavity (Greitz, 1993) both are correlated to the cerebrovascular pulse wave. According to Whedon & Glassey (2009), the “rhythmic brain motion is not primarily endogenous, but is propagated by the cerebrovascular pulse wave,” and synchronous with cardiac systole (Levy et al., 1988; Feinberg & Mark, 1987; Greitz et al., 1992). They go on to state “ . . . systolic arterial expansion causes a pulsatile expansion of the brain, resulting in a piston-like action that compresses the ventricles and propels CSF into the subarachnoid space and the spinal canal (Feinberg & Mark, 1987; Greitz et al., 1992);” and also that “ . . . age-related pathology of the cardiovascular and cerebrovascular circulation, coupled with calcification of the choroid plexus and decreased brain tissue compliance may also contribute to impaired CSF circulation (Rubenstein, 1998);” and finally that “Rubenstein hypothesized that aging can lead to stagnation of the CSF circulation, which in turn may contribute to the development of some age-related dementias (Rubenstein, 1998).” While arterial-driven CSF pulsation might be necessary for proper movement of CSF, there are studies suggesting that excessive pulsation could be harmful, possibly leading to noncommunicating hydrocephalus (O'Connell, 1943; Bering, 1962; Guinane, 1977; Di Rocco et al., 1978; Bateman, 2002; Greitz, 2004), or which might contribute to other harmful conditions (Baumbach, 1996; O'Rourke & Hashimoto, 2007; Alsop et al., 2008; Wahlin et al., 2014).

Zheng et al. (2014) and Xu et al. (2016) showed that another mechanical source for moving CSF, one that is important for the current devices and methods, is generated by head movements. Specifically, they found that CSF movement that was measured at the craniocervical junction increased when subjects rotated their heads, and speculated that three muscles in particular (rectus capitis posterior major, obliquus capitis superior, and obliquus capitis), in the suboccipital triangle, act on the myodural bridge (dense tissue that extends to the cervical spinal dura mater), producing a pumping action that powers CSF circulation. They also showed that subjects experienced no significant increase in heart rate during head rotation, ruling out the explanation that the observed changes in CSF movement were caused by increased pulsation due to muscle activity.

Stivaros & Jackson (2007) described how modern imaging technology has helped discover more about the ability of the incompressible contents of the skull to adapt to the periodic influx of blood during systole and the consequences of failure, which they state “ . . . has been implicated in a wide range of cerebral disorders, including vascular and Alzheimer's dementia, late-onset depression, benign and secondary intracranial hypertension, communicating and normal pressure hydrocephalus, and age-related white matter changes.” Part of the energy from the incoming pulse is absorbed by the expansion of the compliant arterial walls, which return to their shape during diastole, the net result of which is that a relatively constant flow of blood is provided to the capillaries (called the “windkessel effect”). The elastic properties of the arteries, veins, and arachnoid granulations help buffer and more evenly distribute the otherwise extreme pressure changes from the incoming arterial systolic pulses. They also described how imaging technology has helped identify deviant hydrodynamic patterns in arterial blood, venous blood, and CSF in a number of conditions related to NCD including AD, VaD, NPH, communicating hydrocephalus, idiopathic and secondary intracranial hypertension, microvascular angiopathy, and leukoaraiosis, and asserted that the inadequate clearance of waste materials being addressed by other researchers “ . . . could contribute to the onset and severity of cognitive impairment.”

Possible Causes of Glymphatic Dysfunction.

While there are variations and inconsistencies among the various descriptions of glymphatic function in the current literature, the role of arterial pulsation as a driving force for CSF/ISF exchange and transport to the veins is generally emerging as a common thread, as is the notion that inadequate glymphatic function can lead to NCD. Consequently, any deviation from normal arterial pulsation could have implications for proper glymphatic function, so the section below entitled “Adequate Blood Supply and the Importance of Arterial Pulsation” is presumably directly related to possible causes of glymphatic dysfunction. CSF pressure can be too high (e.g., as in hydrocephalus) or too low (low-pressure hydrocephalus—a condition in which enlarged ventricles are accompanied by below-normal CSF pressure). In the healthy brain, CSF pressure changes are associated with CSF movement and the movement of blood in the veins, so abnormally high or low baseline CSF pressure could disrupt the normal movement of waste materials in both the glymphatic and venous systems. Other potential causes for glymphatic dysfunction (e.g., over/under production of ISF/CSF, biochemical changes in the composition of CSF, biochemical failure in the transfer of waste materials to ISF and from ISF to CSF, etc.) are plausible.

Body posture is known to affect the venous pathways used to drain blood from the CNS, but in addition, Lee et al. (2015a) found that movement of CSF in the glymphatic system also is affected by posture. Specifically, they found greater glymphatic movement and the most efficient removal of waste material (including Aβ) from the brains of mice when anesthetized mice were positioned in a lateral posture (on their side) than when in either a prone or supine posture, and speculated that there could be some relationship between body posture while sleeping and NCD. While they only investigated one pathway (from the subarachnoid space to the cervical lymphatics), Simmonds (1952) had previously reported that movement of blood and protein out of the brain to the cervical lymphatics in cats and rabbits was influenced by the animals' posture. Interestingly, and related to the present device and method, they also reported that “Lymph flow was maintained during most experiments by light massage over the deep cervical node and duct.”

Neurological Disorders Associated with Glymphatic Dysfunction.

A number of researchers have suggested that inadequate movement of CSF can contribute to inadequate control of intracranial pressure or inadequate removal of waste materials (e.g., Segal, 2000; Stopa et al., 2001; Silverberg et al., 2003; Johanson et al., 2008). By exploiting data made available from new imaging techniques, computer models have been created which demonstrate how ineffective CSF movement could contribute to neurocognitive deterioration (e.g., Holman et al., 2010 and Gupta et al., 2009, 2010). Shih et al. (2013) showed that occlusion of either single penetrating arterioles or single venules in the cortex of rat subjects degraded cognitive performance. Arteriole pulsation in the confined volume of the perivascular space could produce a significant pumping action that increases the movement of CSF fluid in and out of the subarachnoid space; if the perivascular spaces are dilated, then the same arteriole pulsation would displace the same volume of CSF, but the velocity and pressure would be reduced substantially. Also, if elasticity in the surrounding pia mater absorbed part of the pressure, and that property remained constant for the dilated perivascular spaces, then the volume of CSF moved in/out of the perivascular spaces could be reduced as well (because the overall area of pia mater is increased), and the pumping action would be even further reduced if the elasticity in the walls of the descending arteries is decreased, as would be expected in an elderly person.

### Lymphatic System

Until recently, it was generally accepted that the lymphatic system was not present in the brain. In the rest of the body, lymphatics is an important part in the immune system and among other things, helps with the removal of metabolic wastes. In general, lymph carries waste products, proteins, cellular debris, and bacteria from capillary beds through lymph vessels to lymph nodes, and eventually is emptied into the venous system where it is carried to disposal sites. Like the veins, lymph is moved through lymph vessels in a lock-step manner, with valves preventing backflow. There are two types of lymph vessels and two corresponding types of valves. In most of the body, the initial lymphatic vessels collect ISF and the spaces overlapping cells function as initial valves; flow in these very small vessels presumably is sustained by ongoing lymph formation, and reflux prevented by those initial valves. Lymph passes from the initial vessels to larger collecting vessels, which contain more conventional bicuspid luminal valves which prevent reflux. Lymph flow is sustained in the collecting vessels by a peristaltic-like action, a rhythmical contraction of the walls of the vessel to move lymph from a distal inter-valve section (lymphangion) to the next proximal lymphangion. Venugopal et al. (2004) reported that it is the smooth muscles in the wall of the lymphangion (the section of a lymph vessel between two valves), that is contractile and can help push lymph onward. Researchers have described the pulsing action of the smooth muscles in the lymphatic vessels as playing a pivotal role in maintaining tissue fluid homeostasis and lymph transport, and proposed that their dysfunction “ . . . may be responsible for the impairment of lymph flow that is thought to occur in lymphedema and many inflammatory diseases” (von der Weid & Zawieja, 2004). As in the veins, lymph movement also is induced in the collecting vessels by the application of perpendicular pressure to the vessel walls; pressure sources include external sources (e.g., pushing on the skin), skeletal muscle movements, respiratory movements, and arterial pulsation. There has been an increased interest in the collecting lymphatic system (see Chakraborty et al., 2015—with the research primarily conducted with animals), and a great deal has been learned about the underlying mechanisms and the variables that affect them. Malfunction of the collecting vessels has been associated with a variety of health conditions including lymphedema, metabolic syndrome, obesity, atherosclerosis, and inflammatory bowel disease; and dysfunction has been shown to be positively related to age.

Recently, lymphatic vessels were found in the meninges (dura mater) of the mouse brain for the first time (Louveau et al., 2015 and Aspelund et al., 2015) and subsequently in human and nonhuman primate brains (Absinta et al., 2017), providing valuable new information about the previously unknown process in which ISF and CSF drain from the subarachnoid space to the cervical lymph nodes. Although much has yet to be learned about the lymphatics in the brain, arterial pulsation has been associated with the movement of lymph in the rest of the body. Aspelund et al. (2015) reported a concentration of lymph vessels near the pterygopalatine artery where they exited the skull, so arterial pulsation from nearby arteries might be providing part of the pumping action, serving the role of the muscles in the rest of the body by providing rhythmic external perpendicular forces on the sides of the lymph vessels. In addition, although published six years before the discovery of lymphatic vessels in the brain, Weller et al. (2009) noted evidence for lymphatic drainage from the brain to the cervical lymph nodes in rat brains, and concluded:

“Vessel pulsations appear to be the driving force for the lymphatic drainage along artery walls, and as vessels stiffen with age, amyloid peptides deposit in the drainage pathways as cerebral amyloid angiopathy.” (CAA—a condition associated with NCD) “Blockage of lymphatic drainage of ISF and solutes from the brain by CAA may result in loss of homeostasis of the neuronal environment that may contribute to neuronal malfunction and dementia. Facilitating perivascular lymphatic drainage of amyloid-beta (Abeta) in the elderly may prevent the accumulation of Abeta in the brain, maintain homeostasis and provide a therapeutic strategy to help avert cognitive decline in Alzheimer's disease.”

Possible Causes of Lymphatic Dysfunction.

As with arteries and veins, aging affects the lymphatic vessels. Zolla et al. (2015) reported that the smooth muscles in the walls decrease in contraction frequency and that lymph velocity and pumping action decline in aging rats. In addition, they found that pathogen transport is compromised in older lymph vessels due to an apparent increase in wall permeability, with pathogens escaping into the surrounding tissue in older vessels. As with the valves in the veins, the valves in the lymph vessels can be damaged, missing, or malformed, generally leading to one of two possible outcomes—failure to close properly allowing reflux, or not allowing proper flow through the valve in the correct direction (e.g., by not opening properly and obstructing the flow). Zolla et al. (2015) reported changes in lymphatic valves with aging; specifically, the extracellular matrix surrounding the collector's valves in young rats was reduced in older rats. While little is known about the newly discovered lymph vessels, it seems likely that the same sources of dysfunction will be found to apply to them as apply to lymph vessels in the rest of the body (e.g., hardening of their walls [lymphosclerosis], inflammation, etc.).

Neurological Disorders Associated with Lymphatic Dysfunction.

To date, no research could be found relating the recently discovered lymph vessels/valves to NCD—with the possible exception that poor lymphatic drainage into the cervical lymphatics has been associated with chronic fatigue syndrome (Perrin, 2007). However, both research teams involved in the recent discovery of lymph vessels in the brain (i.e., Aspelund et al., 2015, and Louveau et al., 2015) recognized the potential importance of this finding—that if this system malfunctioned it could potentially contribute to neurological disorders. As discussed by Louveau et al. (2016), discovery of the new lymph system in the CNS introduces a new player in the possible relationship between inadequate protein removal and conditions associated with NCD such as MS and AD. In addition, Kovacs et al. (2016) reported the presence of AP deposits in the human dura of elderly humans, suggesting a possible association between AD and dysfunction in an aging lymphatic system.

Adequate Waste Removal and the importance of Valves

While valves and their possible malfunction were mentioned in the discussions of the venous and lymphatic systems, more detail is presented here because it is argued herein that the presence of valve-like mechanisms in all of the major fluid-based systems involved in waste disposal is a primary source of possible system failure. The known valve mechanisms involved in the removal of waste from the CNS include (1) the jugular vein valves and any venous valves located in the veins that empty into the internal jugular vein (e.g., Shima et al., 1998 reported the presence and location of valves in the middle thyroid vein, facial vein, lingual vein, and superior thyroid vein); (2) valves in the veins in the vertebral venous plexus (primarily the vertebral veins); (3) valves in the newly discovered meningeal lymphatic vessels in the dura mater; (4) the (probable) valve-like functions in the lymphatic capillary endings of the newly discovered lymphatic vessels in the CNS; (5) the vacuolar channels on the arachnoid villi in the subarachnoid space, which serve as one-way valves for CSF (see Tripathi, 1977; Levine et al., 1982; Brinker et al., 2014); (6) any physical structure containing small passageways through which waste-bearing biofluid passes due to a significant decreasing pressure gradient (e.g., the cribriform plate and the blood—brain barrier); and (7) the cilia in the CNS. The last two listed are not true valves, but are included here because they can have valve-like properties and, like the other valves listed, are sites where flow can be impeded and congestion occur.

For each of the listed valves, there are two possible general malfunctions: (a) they are absent, malformed, inverted (possibly due to excessive backpressure), damaged, have an altered/abnormal composition, or are generally malfunctioning in a way that allows the fluid to pass in the wrong direction (reflux); or (b) they are malformed, inverted, etc., in a way that does not allow enough fluid to pass in sufficient amounts in the normal direction. A third possible general type of malfunction (especially in the venous and lymphatic valves), is the situation in which both general types of malfunction are present in a single valve (e.g., a valve that remains partially closed, both allowing regurgitation and blocking forward flow). A malfunction in any of the above sites could have serious implications, i.e., allowing reflux of waste-bearing biofluids or impeding the proper removal of waste-bearing biofluids.

### Adequate Blood Supply and the Importance of Arterial Pulsation

Providing the brain with an adequate supply of oxygen and nutrients obviously is necessary for proper neuronal function. The following sections concentrate on the evidence supporting the apparently necessary role that adequate arterial pulsation plays in the removal of waste, and the variables and conditions likely to affect pulsation.

One of the earliest clinical areas in which the importance of pulsation surfaced was when machines were beginning to be used to provide patients blood during heart bypass surgery. Early findings (most involving lower animals as subjects) were mixed, with some investigators reporting fewer neurological/cognitive issues following bypass surgery during which “artificial” pulsation was added to the bypass process (e.g., Sanderson et al., 1972; DePaepe et al., 1979; Tranmer et al., 1986; Kono et al., 1990; Briceno & Runge, 1994; Onoe et al., 1994; Aykut et al., 2013); and others reporting no difference when pulsation was added (e.g., Henze et al., 1990; Hindman et al., 1995; Murkin et al., 1995; Chow et al., 1997). Baba et al. (2003, 2004) showed that in goats, the addition of pulsation in an artificial heart significantly improved microcirculation (the number of perfused capillaries). Hickey et al. (1983) offered a possible explanation for the variability in outcomes for studies investigating the benefits of adding artificial pulsation to bypass systems—that the outcome probably depends on the form of the artificial pulse being introduced (which usually was not described in any detail). If that is the correct explanation, then taking that comment to the next step, the physical attributes of the artificial pulse added in those studies with positive outcomes (if known) could be compared with those for studies where no benefits were found to move closer to identifying the critical features of the “ideal artificial pulse,” alternatively, the normal, possibly age-adjusted, human heart pulse probably might provide the ideal model.

A number of studies have reported improved microcirculation and organ health/recovery when pulsation was provided during circulatory support compared to no added pulsation (e.g., Nakata et al., 1996; Sezai et al., 1996, 1997, 1999). Undar et al. (2002a, 2002b) found cerebral, myocardial, and renal blood flow to be higher with pulsation, and Undar (2005) reported that the cerebral metabolic rate for oxygen and cerebral oxygen delivery were greater with pulsation. While none of those studies involved human subjects, Sezai et al (2005) reported a significant reduction in systemic inflammation under pulsatile conditions in human patients. In summary, there are numerous studies indicating improved microcirculation and other benefits for pulsatile blood flow compared to non-pulsatile flow. In addition, it should be noted that even if that body of evidence did not exist, it would not necessarily mean that pulsation is not a critical component in circulation; as argued herein (and several others in the past), pulsation could play a major role in the removal of toxic metabolites due to its indirect actions on the movement of venous blood, CSF, ISF, and lymph in the CNS.

Pulsation.

A number of variables can affect the healthy pattern/characteristics of pulsation, some of which are changes in the initial cardiac output (heart stroke volume), reduced integrity of the heart valves—possibly causing a distortion in the natural pulsatile pressure magnitude and pattern, increased peripheral resistance in the circulatory system, altered viscosity of the blood, and changes in the arterial walls. One of the major variables that endangers the integrity of the arterial walls is a reduction in their compliance, which leads to greater arterial stiffness as measured by PP (e.g., Briet et al., 2012). During a blood pulse, the high initial (systolic) pressure from the heart's contraction is transmitted down the arterial tree, with the carotid arteries, the vertebral arteries, and the deep cervical arteries supplying most of the blood to the head. In healthy young arteries, the arterial walls are quite compliant, and a portion of the pulse's pressure wave is absorbed by the momentary expansion of the arterial walls as the systolic pressure peak passes through; during the following diastolic phase, the elastic walls contract, helping sustain pressure in the vessel until the next systole. The overall result is a capacitor-like mechanism called the “windkessel effect” that helps sustain downstream pressure even during diastole. Proper arterial compliance (as well as proper compliance in the other fluid carrying vessels in the CNS) is necessary to ensure not only adequate blood supply but also adequate waste removal.

Numerous variables can affect blood flow in the vascular system. One of the most important variables is systemic vascular resistance, the resistance in the overall circulatory system that works against normal blood flow and must be overcome for proper blood movement. Any increase in systemic vascular resistance slows the velocity and decreases the volume of fluid moved in the system unless compensated by increased cardiac output; and if so, results in increased blood pressure. According to Thurston (1976), the Hagen-Poiseuille equation (which is used to describe fluid flow in a closed tube of constant diameter) must be modified to more accurately describe blood flow. Solving for resistance, a modified form of Thurston's equation is:

$$R = \frac{c * L * η ( δ )}{π * δ * {r}^{⋀} 3} \qquad Equation 1$$

where: R=resistance to blood flow, c=constant coefficient of flow, L=length of the vessel, η(δ)=viscosity of blood in the wall plasma release-cell layering, r=radius of the blood vessel, δ=distance in the plasma release-cell layer

While modeling blood flow is complex and well beyond the scope of the present discussion, Thurston's equation serves as a starting point for identifying the primary variables that alter vascular resistance: resistance is directly related to the length of the vessel, and the viscosity of the blood in the wall plasma release-cell layering (the layer of fluid closest to the walls of the vessel that Thurston proposed to have “modified viscous and viscoelastic properties”); and resistance is inversely related to the radius of the blood vessel and the distance in the plasma release-cell layer.

While length of the vessel is usually discounted because it is remains constant in the human body, there are situations in which it could have a significant influence on systemic vascular resistance. For example, limb amputations decrease the overall vessel length and theoretically decrease resistance and, in contrast, patients undergoing bypass surgery or kidney dialysis have a temporary increase in length (and increased vascular resistance), when vessels are artificially elongated to circulate through external medical devices.

Increasing the blood's viscosity increases systemic vascular resistance, and according to Jeong et al. (2010), blood viscosity is positively correlated to a number of cardiovascular risk factors including hypertension, total cholesterol, VLDL-cholesterol, LDL-cholesterol, triglycerides, chylomicrons, diabetes mellitus, insulin resistance, metabolic syndrome, obesity, cigarette smoking, male gender, and age, and negatively correlated to others including HDL-cholesterol and anemia. In addition to affecting the pulsatility of blood, highly viscous blood is more turbulent, potentially damaging arterial walls and believed to result in plaque production, and possibly explaining why plaque tends to accumulate at bends or bifurcations of the large arteries where turbulence is the greatest.

Arterial Integrity and Pulsation.

Equation 1 also indicates that the diameter of the vessel inversely affects resistance. While vessel diameter is known to be affected by numerous variables, one of the body's primary controls for regulating resistance, and hence blood flow and pulsation is by modification of the diameter of the blood vessels—constricted vessels (resulting from contraction of the smooth muscles in the vessel walls) increase resistance and dilated vessels decrease resistance. Atherosclerosis reduces the diameter of arteries and there is evidence that severe atherosclerosis of the internal carotid artery is related to faster cognitive decline in patients with AD (Silvestrini et al., 2011). Wendell et al., 2012 reported that the rate of subsequently developing dementia in patients in the upper quintile for carotid atherosclerosis was twice that of patients in the lower quintiles. In a recent review, de Bruijn & Ikram (2014) concluded that several cardiovascular diseases and cardiovascular risk factors also are risk factors for AD; their list of established and emerging risk factors included stroke, atrial fibrillation, coronary heart disease, heart failure, intima media thickness, carotid plaque, calcification volume in atherosclerotic plaque, lacunae, white matter lesions, cerebral microbleeds, retinal vessel diameter, hypertension, arterial stiffness (high PP and pulse wave velocity), low blood pressure, type-2 diabetes mellitus, hypercholesterolemia (age-related), smoking, obesity (especially at midlife—Kivipelto et al., 2005), sedentary activity level, inflammation, chronic kidney disease, and thyroid dysfunction. It should be noted that many of the listed risk factors cited for AD involve cardiovascular conditions, which also are risk factors for VaD, reinforcing the possibility that there might be common underlying causal mechanisms.

Blood Pressure.

Cognitive decline has been associated with high blood pressure low blood pressure, and variable blood pressure. While high blood pressure is commonly cited as a risk factor for various conditions associated with NCD, it does not appear to be a sole causative factor. Elmstahl & Rosen (1997) showed that a drop in systolic BP while standing up (orthostatic hypotension) was associated with subsequent cognitive decline (measured 5 years later). Novak et al. (1998) showed that some patients with orthostatic hypotension have significantly reduced autoregulation of cerebral blood flow. Verghese et al., 2003 conducted a large longitudinal study of people over the age of 75 and found that low diastolic blood pressure was significantly associated with a higher risk of developing AD but not other types of dementia. Periods of reduced blood pressure are likely to produce poor blood perfusion in the brain, and the resulting hypoxia could trigger a chain of events that leads to the accumulation of Aβ protein, as hypothesized by Sun et al. (2006) and Zhang et al. (2007).

Neurological Disorders Associated with Cardiovascular Dysfunction.

In general, aging produces lower vascular compliance in the elderly (i.e., stiffer arteries as indicated by higher PP, pulse wave velocity—PWV—the estimated speed of the pulsing blood, and the pulsatility index—PI—the difference between the peak systolic and end diastolic velocities divided by the mean flow velocity and reported to be analogous to PP, Roher et al., 2011b). In addition, various health conditions can affect pulsatility, for example, thickened/stiffened artery walls (arteriosclerosis) as well as arterial walls stiffened by accumulated plaque (atherosclerosis) both reduce arterial compliance and increase overall resistance. Henry-Feugeas (2009) described how the windkessel effect decreases with age (e.g., due to arteriosclerosis), causing greater PP, and suggested that the current data indicate that the primary medical target late in life should not be AD, but cerebrovascular dysfunction.

Several researchers have reported that increased arterial stiffness is positively correlated to cognitive decline or degree of dementia: Hanon et al., 2005; Scuteri et al., 2005; Lee et al., 2006; Scuteri et al., 2007; Muller et al., 2007; Vicenzini et al., 2007; Waldstein et al., 2008; Kearney-Schwartz et al., 2009; Elias et al., 2009; Stefani et al., 2009; Pase et al., 2010; Mitchell et al., 2011; Roher et al., 2011b; Nation et al., 2012; Yaneva-Sirakova et al., 2012; and Pase et al., 2013. However, some researchers have not found that relationship: Poels et al., 2007 and Dhoat et al., 2008. Still other studies have reported the reverse relationship (i.e., greater stiffness was related to better cognitive performance): Roher et al., 2012 and van Bruchem-Visser et al., 2009. To further complicate matters, Qiu et al., 2003 studied PP measures in subjects with AD and in subjects with all types combined and reported a complex relationship: women showed increased risk of dementia for either very low or very high PP, while men with low PP showed greater risk of dementia. In addition, an important study by Wahlin et al. (2014) showed that intracranial pulsatility of CSF (generally believed to be determined by blood pulsation), was negatively related to brain volume and positively related to ventricle size; they concluded that their results support the hypothesis that “excessive cerebral pulsatility damages the cerebral microcirculation, leading to brain atrophy in elderly persons.”

Kalaria et al. (2012) reported that cardiovascular risk factors for AD included arterial stiffness, dyslipidaemia, adiposity, endothelial degeneration, and blood-brain barrier dysfunction. The brains of patients with AD and other dementias have been noted to have abnormal microvascular structures including swelling, kinking, twisting, and looping (Hassler, 1967 and Zarow et al., 1997), and these abnormalities have been linked to altered blood flow and Aβ accumulation (Meyer et al., 2008). In addition, a common characteristic of patients with AD is poor blood flow in the brain (de la Torre & Mussivand, 1993 and Bateman et al., 2006). Reitz et al. (2007) reported that compared to controls, patients with AD have a higher incidence of aortic valve thickening, aortic valve regurgitation, left ventricular wall motion abnormalities, left ventricular hypertrophy, and reduced ejection fraction, while patients with VaD were more likely to have aortic valve regurgitation, mitral valve thickening, and tricuspid valve regurgitation. De la Torre (2006) suggested that non-genetic AD is primarily a vascular disease caused by hypoperfusion (poor blood supply) resulting in protein synthesis defects that result in the accumulation of Aβ plaque and neurofibrillary tangles.

Research has shown that adequate pulsation is required for the removal of AP and other solutes, and that adequate pulsation diminishes with age (e.g., Hawkes et al., 2011; Kress et al., 2014). It also has shown that cerebrovascular pulsation produces fluid movement in the perivascular spaces of the brain (Iliff et al., 2013b) and spinal cord (Martin et al., 2012), that is believed to be important for the interactive exchange of waste materials such as AP (e.g., Hawkes et al., 2011). Using M M, Selvarajah et al. (2009) found significant differences in CSF pulsatility in patients with many versus few risk factors for cerebrovascular disease; they attributed the difference to decreased arterial compliance due to thickened arterial walls in the high-risk group.

RRobles et al. (2014) reported that both bradycardia (slow heart rate) and low systolic blood pressure were significantly related to frontotemporal dementia (FTD). They did not report any significant findings for diastolic pressure, so it would appear that PP was lower in the FTD group than in the control group (i.e., FTD patients had both slower heart rates and lower PP).

### Other Implicated Patient Populations

In addition to patients formally diagnosed with dementia, there are a variety of NCD conditions related to the normal supply of blood to the brain and the normal movement of ISF, lymph, or CSF from the brain. This includes patients with cardiac valvular disease (e.g., aortic stenosis, mitral stenosis, valvular regurgitation); patients with heart conditions (e.g., cardiac arrhythmia, congestive heart failure, decreased cardiac output); patients with inefficient artificial mechanical heart valves; patients undergoing or following bypass surgery; patients with blood/lymph vessel or circulation inefficiency (e.g., atherosclerosis, arteriosclerosis, venous sclerosis, lymphedema, chronic cerebrospinal venous insufficiency, internal jugular vein valve incompetence or blockage, neurocardiovascular instability, chronic obstructive pulmonary disease, primary pulmonary hypertension, primary exertional headache, transient monocular blindness, chronic hypotension, bradycardia, moyamoya disease); patients in which blood-flow dynamics are significantly altered (e.g., people undergoing kidney dialysis treatment, or placed in altered gravitational fields); patients with neurocardiovascular instability (see the review by O'Callaghan & Kenny, 2016—including hypotension, carotid sinus hypersensitivity, postprandial hypotension, and vasovagal syncope); patients with damaged or altered circulatory physiology due to trauma (e.g., traumatic brain injury, chronic traumatic encephalopathy) and patients with conditions related to poor CSF circulation (e.g., glaucoma, communicating and noncommunicating hydrocephalus, idiopathic or secondary intracranial hypertension, people with spontaneous or induced carotid sinus syndrome). It should be noted that dividing the various conditions into two groups—those involving supply issues and those involving waste removal issues is problematic because, under the current thesis, one or both situations might be implicated; for example, inadequate pulsatility might produce poor blood perfusion (blood supply) while also resulting in poor CSF, lymph, venous blood movement (waste removal).

Patients with Faulty Heart Valves.

Studies have reported relationships between/among: (a) heart valve disease and cognitive decline (e.g., de la Torre, 2006); (b) aortic valve thickening, regurgitation, and AD (Reitz et al., 2007); (c) poor transmittal flow efficiency and AD (Belohlavek et al., 2009); and (d) post mortem findings of valve damage and AD (Corder et al., 2005). Adequate arterial pulsation has been argued above to be an instrumental component not only in supplying the brain with oxygen and nutrients, but also in the subsequent removal of waste materials. Consequently, any heart-related problem/deviation affecting arterial pulsatility could have implications for both blood perfusion (i.e., supply) and for the proper removal of waste from the CNS; either possibly affecting cognition. For example, problems with either of the major heart valves such as stenosis (the valve does not open enough), could affect the magnitude of blood pushed into the arteries or the fluid dynamics of the produced pulse (i.e., pulsatility, which has been shown above to be related to ISF/CSF exchange and waste removal).

Patients with Artificial Heart Valves.

Another potentially impacted population comprises patients who have received an artificial heart valve (AHV). Decreased cognitive performance (post-operative cognitive dysfunction—POCD), has been reported for AHV patients (e.g., Deklunder et al., 1998; Grimm et al., 2003; Uekermann et al., 2005; Zanatta, 2012). According to Deklunder et al. (1998), transcranial Doppler examination of the middle cerebral artery indicates that patients with an AHV have elevated rates of high-intensity transient signals (HITS—suspended emboli which are solid or gaseous) in their cerebral circulation (e.g., Deklunder et al., 1993; Rams et al., 1993; Dauzat et al., 1994; Braekken et al., 1995; Thoennissen et al., 2005). Such HITS are not produced by a normally functioning heart. Laas et al. (2003) showed that the incidence of HITS depends in part on the design of the artificial heart valve and its orientation. The incidence HITS has been shown to increase with myocardial contractility and heart rate (see Deklunder et al., 2000), and their presence has been documented in a number of other medical procedures (e.g., cardiac catheterization, percutaneous coronary angioplasty, left ventricular assist devices, and general cardiac surgery—see Dittrich & Ringelstein, 2008), some of which also have been associated with subsequent cognitive degeneration. POCD also has been associated with patients who have had open-heart bypass surgery (e.g., Savageau et al., 1982; Smith et al., 1986; Shaw et al., 1986; Padayachee et al., 1987; Roach et al., 1996, Taylor, 1998; Diegeler et al., 2000; Dutta & Ashton, 2001). An alternative explanation for the apparent relationship between HITS and subsequent NCD is that it is not the presence/number of HITS/microbubbles that is directly causing NCD, but rather that the number of HITS is inversely related to the quality of heart valve (i.e., poorly functioning AHVs not only produce more HITS but also affect pulsatility and the quality of the pulse being generated.

Patients with Atrial Fibrillation and Pacemakers.

Atrial fibrillation (AF) is characterized by rapid and chaotic beating of the heart and is a leading cause of embolic stroke. In addition to irregularity, Lee et al. (2015b) reported that “Decreased pulsatility strength in the left atrium was associated with recurrent atrial fibrillation.” Rawles & Rowland (1986) described a pattern of atrial fibrillation in which stronger/longer pulses tended to follow longer inter-beat periods; the stronger/longer pulses were, in turn, followed quickly by a weaker/shorter pulse. Some investigators have reported a relationship between AF and cognitive decline (e.g., Ott et al., 1997; Bunch et al., 2010; de Bruijn et al, 2015; Bunch et al., 2016). Other studies finding no such relationship tended to use elderly subjects (Rastas et al., 2007; Park et al., 2007; Peters et al., 2009; Marengoni et al., 2011), and a fairly recent meta-analysis concluded that a relationship does exist (i.e., Kalantarian et al., 2013).

By definition, AF affects the pulsation pattern, and pacemakers are sometimes prescribed for patients with AF. No evidence could be found suggesting that pacemakers are causally related to NCD—indeed, of three studies found, one reported improved cerebral brain flow and verbal intelligence after pacemaker implantation (Koide et al., 1994), and two others reported no significant measurable effects (Rockwood et al., 1992; Gribbin et al., 2005).

Patients with Parkinson's Disease.

In addition to impaired motor performance, PD also is related to NCD (e.g., Biundo et al., 2016), and patients with PD can develop more severe forms of NCD (i.e., PDm). A hallmark for PD, PDm, and DLB is the presence of Lewy bodies (structures containing alpha-synuclein protein) in the brain—especially in the brainstem and midbrain. Gross et al. (1972) reported normal recumbent blood pressure and normal response to the Valsalva maneuver in a group of Parkinsonian patients, but there was a significantly greater fall in mean blood pressure upon tilting when compared to age-matched controls subjects. Also, the results from a study conducted by Corvol et al. (2007) can be interpreted to suggest that patients with more serious heart-valve problems had more advanced levels of PD.

Patients with Thyroid and Parathyroid Conditions.

There usually are four parathyroid glands and they can be found at various locations, but typically on the thyroid gland in the neck. One cause of hyperparathyroidism is adenoma—the development of a benign tumor that can cause the parathyroid to overproduce parathyroid hormone (PTH). There have been reports of hyperparathyroidism being associated with dementia, which—following surgical removal of the tumor—was dramatically reduced (e.g., Watson & Marx, 2002; Papageorgiou et al., 2008; Chou et al., 2008). There is a similar report of remarkable recovery following parathyroidectomy in a patient with psychotic symptoms and short-term memory loss (Papa et al., 2003). There are a host of possible explanations for such remarkable outcomes (especially with the endocrine system involved); however, one explanation is that because of the physical proximity of the thyroid and parathyroid glands to the carotid arteries and jugular veins, the increased volume of mass from the tumors could have interfered with normal pulsatile input to the brain (i.e., pressure was exerted on the carotids), or could have blocked the normal evacuation of blood from the brain (i.e., pressure was exerted on the jugular vein), or both. The latter possibility (occluded jugular vein) also indirectly could affect pulsatility because increased resistance from a blocked vein could reduce the pressure gradient required for moving blood out of the brain, by activating homeostatic mechanisms which increase pulsatility.

Patients with Intracranial Hypertension and Hydrocephalus.

The lymphatic system has been proposed to be a major disposal path for CSF; for example, Kida et al. (1993) and Fard et al. (2007) showed that high-pressure hydrocephalus can develop if the lymph system is not functioning properly. Abnormally high CSF pressure (intracranial hypertension) can be caused by a number factors (e.g., brain tumors, encephalitis, head injury, hydrocephalus, hypertensive brain hemorrhage, intraventricular hemorrhage, meningitis, subdural hematoma, status epilepticus, stroke), and can restrict blood flow to the brain. Increased CSF pressure also might be caused by any factor that reduces the flow of ISF, CSF, lymph, or blood along the waste-elimination paths, such as narrowed noncompliant passageways or damaged valves that function more like dams than gateways (e.g., internal or external jugular vein valves, vertebral vein valves, other cranial or cervical veins, lymphatic valves, valve-like mechanisms in the perivascular space, arachnoid ventricular cilia). In support of this possibility, studies have shown that: (a) jugular vein valve insufficiency is more likely to be present in patients with intracranial hypertension (e.g., Nedelmann et al., 2009); (b) cerebral blood flow is reduced in patients with communicating hydrocephalus (Tanaka et al., 1997; Egnor et al., 2002; Owler et al., 2004); and (c) there is a high incidence of cardiovascular disease and AD among patients with idiopathic normal-pressure hydrocephalus (Bech-Azeddine, 2007).

Patients with Traumatic Injuries Involving Extreme External Forces.

Traumatic events involving direct blows to the head obviously can cause immediate and significant neurological damage. However, there also are situations in which neurocognitive effects from a traumatic event continue longer than expected or even occur later. For example, there is a relationship between head trauma and PDm, but typically only after a period of time has passed (often many years). Similarly, according to Stemper & Pintar (2014), there is clinical evidence suggesting that a concussion due to an explosive blast produces different outcomes than a more standard concussion due to head rotation acceleration. While still controversial, there have been indications that concussions from a blast are more likely to be followed by longer-term mental and physical issues such as post-traumatic stress disorder and depression (Rosenfeld & Ford, 2010). There also are reports of increased incidence for longer-term NCD conditions following blast-related concussions (e.g., McKee & Robinson, 2014). Goldstein et al. (2012) conducted a postmortem study of the brains of US military veterans exposed to blasts and found evidence of chronic traumatic encephalopathy (CTE), a tau protein related NCD condition. They also found that exposing mice to a single blast was “sufficient to induce early CTE-like neuropathology.”

One explanation for some of the more unusual effects of blast concussion deals with the fact that a “volumetric blood surge moves through blood vessels from the high-pressure body cavity to the low-pressure cranial cavity, causing damage to tiny cerebral blood vessels and the blood-brain barrier” (Chen et al., 2013). In the current discussion, it is suggested that not only is there blood reflux, but that venous blood, CSF, and lymph all are forced backwards under high pressure in a direction that goes against their normal flow, possibly damaging vessels or valvular mechanisms, and impeding the subsequent removal of extracellular proteins and metabolic waste material from the brain. In support of this notion, Xiong et al. (2014) reported that TBI inflicted on mice resulted in a decrease in cilia in the ventricles, reduced CSF flow, and reduced nutrient exchange. In that study, there was apparently complete recovery within 30 days, however, depending on the forces and systems involved, such damage might not be evident until years later, when the arterial pulsation that powers most of the disposal mechanisms has been reduced due to the ageing process or inactivity. The implication for this discussion is that any extreme increase in pressure causing biofluids to move forcefully into the neck and skull (such as a blast or excessive Valsalva maneuver), could potentially damage any one-way valves in those pathways (see the above examples of one-way valves). In addition, the effects of a diminished (but not destroyed) capacity for normal waste removal might not show up until after years of accumulation, not unlike the long-term buildup of Aβ plaque in AD patients which is known to occur over an extended period of time.

Patients with Heart Failure.

Increased jugular vein pressure is a well-known sign of congestive heart failure (CHF), and Pellicori et al. (2014) reported significant increases in the diameter of the internal jugular veins of patients with CHF (unfortunately, there was no mention of the condition of the valves in those veins). CHF also has been proposed to be a risk factor for AD (e.g., Qiu et al., 2006), and declining cognitive performance in general (e.g., Cacciatore et al., 1998; Zuccala et al., 2001; Trojano et al., 2003; Hoth et al., 2008; Sauvé et al., 2009; Dardiotis et al., 2012). Both the direct damage due to poor blood circulation in the brain (e.g., Georgiadis et al., 2000; Pullicino et al., 2001), and possible indirect damage due to the resulting inadequate removal of Aβ (e.g., Cermakova et al., 2015), have been implicated as possible causes for the relationship between CHF and NCD. As pointed out by Muqtadar et al. (2012), if the underlying cause linking CHF to NCD is hypoperfusion, then there is reason to believe that its effects can be reversed by cardiac interventions.

Patients Undergoing Certain Medical Procedures: Stints, Heart Bypass Surgery and Dialysis.

As pointed out by Furukawa et al. (2010), the internal jugular veins are a common catheterization site for “ . . . hemodynamic monitoring, long-term administration of fluids, antibiotics, total parenteral nutrition, chemotherapeutic drugs, and hemodialysis,” and although maintaining the competency of the internal jugular vein valves is clinically important, “ . . . many physicians remain unaware of the IJV.” Wu et al. (2000) showed that the internal jugular vein valves can be significantly damaged by medical cannulation and catheterization procedures, and that while injury occurred in both proximal and distal catheterization, its incidence was higher after proximal catheterization. Imai et al. (1994) expressed concern about possible injury to the internal jugular vein valve during common cannulation and catheterization medical procedures.

In a discussion of the long-lasting controversy over whether pulsatility is important for heart-lung machines and cardiac assist devices, Ji & Undar (2007) pointed out that one of the critical questions is the extent that external pulsation is transmitted to the microcirculation in the capillary beds, and that the answer to that question was still controversial. They concluded that there was evidence that pulsation at the capillary level could be induced (Intaglietta et al., 1970; Lee et al., 1994) and that “Most authors agree that the pulse has a major role in the movement of lymph, prevention of edema and sludging in capillaries, and maintenance of flow in the capillaries to prevent shunting” (citing Mavroudis, 1978).

Patients Susceptible to Syncope and Falling.

Falling is one of the most common, dangerous, and costly health/safety issues affecting the elderly. There are numerous causal factors associated with falling, but presyncope and syncope often are involved, and they can be triggered by changes in blood pressure. The carotid sinus is a site that monitors blood pressure in one of the primary vessels supplying the brain; it is located at the bifurcation of external and internal carotids (where the carotid artery divides into the external and internal carotids). The carotid sinuses contain baroreceptors which are sensitive to mean blood pressure, rate of blood-pressure change, and PP. The carotid sinus normally helps monitor and regulate blood supply to the brain, overall blood pressure, and cardiac output. Low mean blood pressure or a sudden drop in blood pressure in the carotid sinus causes its walls to constrict, triggering the baroreceptors which send messages via the carotid sinus nerve to the medulla, resulting in decreased parasympathetic activity and increased sympathetic activity. In the cardiovascular system, this chain of events leads to increased (restored) blood pressure due to (1) vasoconstriction of the blood vessels which increases systemic vascular resistance, and (2) increased cardiac output due to increased heart rate and increased strength of heart-muscle contraction. In similar fashion, any sensed high or suddenly increased blood pressure produces the opposite outcome through a parallel series of events. Orthostatic hypotension (syncope or presyncope experienced while a person is in the act of sitting up or standing up), usually is attributed to the redistribution of blood to the lower extremities, resulting in reduced blood supply to the brain. While the carotid sinus reflex works to correct such conditions, in some situations/individuals it cannot act quickly enough to restore an adequate blood supply, and the reduced blood supply to the brain produces presyncope and syncope. This appears to be especially true for people who are hypotensive to begin with, because syncope and falling has a high incidence in patients who are hypotensive (e.g., Campbell et al., 1989).

Another event triggering syncope is when the carotid sinus is overly sensitive to increases in blood pressure (a condition called carotid sinus hypersensitivity—see Klabunde, 2016). People who are hypotensive also are more prone to episodes of syncope due to this condition—i.e., an increase in pressure reduces cardiac output and increases arterial resistance—possibly because they have low blood pressure to begin with (i.e., their current average historical level is low, so it doesn't take as much increase in blood pressure in the carotid sinus to elicit the reflex). Importantly, the increase in pressure in the carotid sinus can be due to an external pressure source. For example, especially in elderly males who are hypotensive and in whom the baroreceptors are overly sensitive, the application of external pressure to the carotid sinus can result in dizziness, fainting, and slowing of the heart (and in extreme cases, cardiac arrest). The classic clinical example is the elderly gentleman who faints while shaving because of putting external pressure on the carotid sinus.

Because some embodiments of the present invention involve the application of external force to the carotid artery, extreme care must be taken to screen patients who are susceptible to syncope, or who have a history of carotid sinus hypersensitivity, and use great caution in any procedures involving the application of pressure to the neck—see the section on Health and Safety Risks and Precautions. Even with this important safety consideration, there still are potential advantages of new alternative treatment involving applying pressure to the carotid(s) if treatment is implemented correctly. Specifically, if additional pressure is introduced in a controlled setting at a low level and then gradually increased over time, then in the long run patients potentially could benefit from increased perfusion and removal of waste materials (due to gradually increased pulsatility), and an increased average historical pressure (because the mean pressure has been gradually increased). Given that conditioning occurs and the average historical level of pressure increases, then (a) the probability of syncope and orthostatic hypotension could decrease because more blood is being supplied to the brain in general (so it is less susceptible to sudden decreases), and (b) the chance of sinus hypersensitivity could be reduced because the average historical level has been raised, making it less likely that a spurious external force or other increase in blood pressure would trigger the reflex. This line of reasoning is strengthened by the fact that one of the most effective treatments for extreme syncope is a pacemaker (Kenny et al., 2001—although there usually are other patient conditions that further justify that option), possibly because pacemakers standardize and increase the overall blood pressure (i.e., the average historical level is increased). In addition, the devices/methods described herein might be used more directly help prevent orthostatic hypotension by monitoring the blood pressure and changes in orientation in real time, and then applying pressure to the arteries and veins when, for example, a person rises quickly (helping sustain adequate blood pressure in the carotid sinuses and brain).

In summary, over the past two decades much has been learned about the variables and conditions related to NCD, but the specific cause or sequence of events that leads to NCD is still unknown. There is increasing evidence: (a) linking the vascular system to NCD; (b) linking inadequate removal of waste materials from the CNS with NCD; (c) implicating arterial pulsation as a critical factor in moving waste-bearing biofluids (i.e., venous blood, CSF, ISF, and lymph); and (d) identifying the various ways the normal waste elimination processes can be disrupted. While medical researchers continue to search for pharmacological solutions that interrupt/reverse the processes that lead to NCD, there is a need for other devices/methods which can apply more direct hydrodynamic interventions, such as the noninvasive methods described herein. The present general method involves applying external stimulation (pressure, vacuum, or electrical stimulation) at specific targeted body locations based on different patient symptomology with the goal of: (a) improving the supply and perfusion of fresh blood in the brain's parenchyma of patients with low pulsatility and hypoperfusion by applying stimulation at specific target sites to increase arterial pulsatility; (b) improving ISF/CSF exchange and movement in patients with poor CSF movement by increasing arterial pulsatility, (c) improving the removal of waste material from the brain parenchyma by augmenting the movement of blood in the veins and lymph in the lymph vessels by applying external pressures to those vessels, (d) reducing/reshaping pulsatility in patients with excessive PP in an attempt to sustain an adequate blood supply while reducing peak systolic pressure, (e) providing an “artificial valve” function in veins and lymph vessels by applying external forces, and (f) providing an artificial valve function in arteries, veins, and lymph vessels when patients rise suddenly by external momentary pressure to those vessels when significant postural or blood pressure changes are detected. Finally, the literature suggests great diversity among the various conditions associated with NCD, and some of that variance might be explained by individual differences in the affected arterial pathways and disposal paths/mechanisms (or both); if so, then unlike pharmaceutical solutions, the present invention offers the important advantage of being customizable with respect to targeted vessels/sites and the countermeasure(s) applied at each site.

### REFERENCES

Absinta et al. (2017). Elife, October 3; 6. pii: e29738.
Aldrich et al., (2016). Journal of Innovative Optical Health Sciences, 10(2), 1-14.
Al-Omari and Rousan, (2010). International Angiology, 29, 115-120.
Alsop et al., (2008). Neuroimage, 42, 1267-1274.
Arbel-Ornath et al., (2013). Acta Neuropathology, 126(3), 353-364.
Aspelund et al., (2015). J. Exp. Med., 212, 991-999.
Aykut et al., (2013). Journal of Cardiovascular Disease Research, 4, 127-129.
Baba et al., (2003). Artificial Organs, 27, 875-881.
Baba et al., (2004). American Society for Artificial Internal Organs Journal, 50, 321-327.
Baracchini et al., (2011). Annals of Neurology, 69, 90-99.
Bateman, (2002). Neuroradiology, 44, 740-748.
Bateman, (2003). Neuroradiology, 45, 65-70
Bateman, (2004). Medical Hypotheses, 62(2), 182-187.
Bateman et al., (2016). Fluids Barriers CNS, 13, 18.
Bateman et al., (2005). Neuroradiology. 47(10), 741-748.
Bateman et al., (2006). J Clinical Neuroscience, 13, 563-568.
Baumbach, (1996). Hypertension, 27, 159-167.
Bech-Azeddine et al., (2007). Journal of Neurology, Neurosurgery, & Psychiatry, 78(2), 157-61.
Beggs et al., (2016). PLoS ONE, 11(5), e0153960. doi:10.1371/journal.pone.0153960.
Beggs et al., (2014a). Journal of Magnetic Resonance Imaging, 40(5), 1215-1222.
Belohlavek et al., (2009). Journal of Ultrasound Medicine, 28, 1493-1500.
Bergan et al., (2008). Journal of Vascular Surgery, 47(1), 183-192.
Bering, (1962). Journal of Neurosurgery, 19, 405-413.
Biundo et al., (2016). Nature Partner Journals Parkinsons Disease, September 1; 2:16018.
Braekken et al., (1995). Stroke, 26, 1225-1230.
Briceno and Runge, (1994). Journal American Society for Artificial Internal Organs, 40, M344-M350.
Briet et al., (2012). Kidney International, 82, 388-400.
Brinker et al., (2014). Fluids and Barriers of the CNS, 11:10, 1-16.
Bunch et al., (2010). Heart Rhythm, 7(4), 433-437.
Bunch et al., (2016). Journal of the American Heart Association, 5(7), pii: e003932. doi: 10.1161/JAHA.116.003932.
Burke et al., (2002). Circulation, 105, 419-424.
Cacciatore et al., (1998). Journal of the American Geriatrics Society, 46, 1343-1348.
Campbell et al., (1989). Journal of Gerontology, 44(4), M112-117.
Carare et al., (2008). Neuropatholology and Applied Neurobiology, 34, 131-144.
Carare et al., (2013). Neuropathology and Applied Neurobiology, 39(6), 593-611.
Carare et al., (2014). Brain, Behavior, and Immunity, 36, 9-14.
Cermakova et al., Journal of Internal Medicine, 277(4), 406-425.
Chakraborty et al., Seminars in Cell & Developmental Biology, 38, 55-66.
Chetelat et al., (2002). Neuroreport, 13, 1939-1943.
Chen et al., (2013). Journal of Neuropsychiatry & Clinical Neurosciences, 25(2), 103-110.
Chou et al., (2008). Surgery, 143(4), 526-532.
Chow et al., (1997). Perfusion, 12, 113-119.
Chung et al., (2014). Journal of Alzheimers Disease, 39(3), 601-609.
Cirovic et al., (2003). Aviation, Space, and Environmental Medicine, 74, 125-131.
Coloma et al., (2016). Journal of Mathematical Biology, 73(2), 469-490.
Cooper and Hainsworth, (2001). Experimental Physiology. 86(5), 677-681.
Corder et al., (2005). Journal of Biomedicine and Biotechnology, 2005, 189-97.
Corvol et al., (2007). Archives of Neurology, 64(12), 1721-1726.
Cushing, (1927). Lancet, 209, 851.
Dardiotis et al., (2012). Cardiology Research and Practice, 2012, Article ID 595821, 1-9, URL dx.doi.org/10.1155/2012/595821.
Dauzat et al., (1994). Journal of Ultrasound in Medicine, 13, 129-135.
de Bruijn et al., (2015). JAMA Neurology, 72(11), 1288-1294.
de Bruijn and Ikram, (2014). BioMed Central Medicine, 12:130, URL biomedcentral.com/1741-7015/12/130.
de la Torre, (2006). Neurological Research, 28(6), 637-644, de la Torre, (2009). Journal of Stroke & Cerebrovascular Diseases, 18(4), 319-328 de la Torre and Mussivand, (1993). Neurological Research, 15, 146-153.
Deklunder et al., (1993). Circulation, 8, 1-223. Abstract.
Deklunder et al., (2000). Texas Heart Institute Journal, 27(3), 236-239.
Deklunder et al., (1998). Stroke, 29(9), 1821-1826
Di Rocco et al., (1978). Experimental Neurology, 59, 40-52.
Diegeler et al., (2000). Annals of Thoracic Surgery, 69, 1162-1166.
DeMattos et al., (2002). Science, 295, 2264-2267.
DePaepe et al., (1979). In: A Propos du Dèbit Pulse. Belgium: Cobe Laboratories.
Dhoat et al., (2008). Age and Ageing, 37, 653-659.
Dittrich and Ringelstein, (2008). Stroke, 39(2), 503-511.
Doepp et al., (2010). Annals of Neurology, 68, 173-183.
Doepp et al., (2004). Neuroradiology, 46, 565-570.
Doepp et al., (2006). Neurological Research, 28(6), 645-649.
Dutta and Ashton, (2001). Journal of the Royal Society of Medicine, 94(4), 188-189.
Egnor et al., (2002). Pediatric Neurosurgery, 36, 281-303.
Elias et al., (2009). Hypertension, 53, 668-673.
Elmstahl and Rosen, (1997). Dementia and Geriatric Cognitive Disorders, 8, 180-187.
Ethell, (2014). Journal of Alzheimers Disease, 41(4), 1021-1030.
Fard et al., (2007). Journal of Theoretical Biology, 248, 401-410.
Farkas and Luiten, (2001). Prog Neurobiology, 64(6), 575-611.
Feinberg and Mark, (1987). Radiology, 163(3), 793-799.
Foltz, (1984) In: Shapiro K, Marmarou A, Portnoy H (eds) Hydrocephalus. Raven, N.Y., 337-362.
Foltz and Aine, (1981). Surgical Neurology, 15, 283-293.
Franceschi et al., (1995). Dementia, 6, 32-38.
Furukawa et al., (2010). Internet Journal of Human Anatomy, 2(1), 1-5.
Georgiadis et al., (2000). European Heart Journal, 21, 407-413.
Gisolf et al., (2004). Journal of Physiology, 560(Pt 1), 317-327.
Goldstein et al., (2012). Science Translational Medicine, 4(134), 134ra60. doi: 10.1126/scitranslmed.3003716.
Gorelick et al., (2011). Stroke, 42, 2672-2713.
Graban et al., (2009). Journal of Neurological Science, 283, 116-118.
Grady et al., (1986). Journal of Neurosurgery, 65(2), 199-202.
Greitz, (1993). Acta Radiology Suppl, 386, 1-23.
Greitz, (2004). Neurosurgical Review, 27(3), 145-165.
Greitz et al., (1992). Neuroradiology, 34(5), 370-380.
Gribbin et al., (2005). Heart, 91, 1209-1210.
Grimm et al., (2003). European Journal of Cardio-thoracic Surgery, 23, 265-271.
Gross et al., (1972). The Lancet, 299(7743), 174-176.
Guinane, (1977). Journal of Neurological Science, 32, 1-8.
Guo et al., (2013). Cell, 154(1), 103-117.
Gupta et al., (2009). Journal of Biomechanical. Engineering, 131(2), 021010. (doi:10.1115/1.3005171).
Gupta et al., (2010) Journal of the Royal Society Interface., 7(49), 1195-1204.
Hadaczek et al., (2006). Molecular Therapy, 14, 69-78.
Hallen et al., (2010). Journal of Pain & Symptom Management, 40(1), 95-101.
Hanon et al., (2005). Stroke, 36, 2193-2197.
Harmon and Edwards, (1987). American Journal of Cardiovascular Pathology, 1(1), 51-54.
Hassler, (1967). Acta Neuropathology (Berl), 8, 219-229.
Hatt et al., (2015). American Journal of Neuroradiology, 36(10), 1971-1977.
Hawkes et al., (2011). Acta Neuropathology, 121(4), 431-443.
Henry-Feugeas, (2009). Journal of Neurological Science, 283(1-2), 44-48.
Henze et al., (1990). Thoracic Cardiovascular Surgery, 38, 65-68.
Heun et al., (1994). Dementia, 5, 327-333.
Hickey et al., (1983). Annals of Thoracic Surgery, 36(6), 720-737.
Hindman et al., (1995). Anesthesiology, 82, 241-250.
Ho et al., (2002). Journal of Trauma, 53(6), 1185-1188.
Holman et al., (2010). Journal of the Royal Society Interface, 7(49), 1195-1204.
Hoth et al., (2008). Cognitive and Behavioral Neurology, 21(2), 65-72.
Hulme and Cooper, (1976). Intracranial Pressure III. Berlin: Springer-Verlag, 259-263.
Iliff and Nedergaard, (2013). Stroke, 44(6 suppl 1), S93-S95.
Iliff et al., (2013a). Journal of Clinical Investigation, 123, 1299-1309.
Iliff et al., (2012). Science Translational Medicine, 4(147): 10.1126/scitranslmed.3003748.
Iliff et al., (2013b). Journal Neuroscience, 33, 18190-18199.
Imai et al., (1994). Anesthesia & Analgesia, 78(6), 1041-1046.
Intaglietta et al., (1970). Microvascular Research, 2, 462-473.
Jeong et al., (2010). Cardiovascular Drugs and Therapy, 24(2), 151-160.
Ji and Undar, (2007). Perfusion, 22(2), 115-119.
Johanson et al., (2008). Cerebrospinal Fluid Res., 5:10.
Juurlink, (2015). Current Neurovascular Research, 12, 199-209.
Kalantarian et al., (2013). Annals of Internal Medicine, 158, 338-346.
Kalaria et al., (2012). Journal of Neurological Sciences, 322(1-2), 141-147.
Kearney-Schwartz et al., (2009). Stroke, 40(4), 1229-1236.
Kenny et al., (2002). Annals of New York Academy of Sciences, 977, 183-195.
Kenny et al., (2001). Journal of the American College of Cardiology, 38(5), 1491-1496.
Kida, (2014). Rinsho Shinkeigaku, 54(12), 1187-1189.
Kida et al., (1993). Neuropathology and Applied Neurobiology, 19, 480-488.
Kitano et al., (1964). Journal of Nuclear Medicine, 5, 613-625.
Kivipelto et al., (2005). Archives of Neurology, 62(10), 1556-1560.
Klabunde, (2016). Cardiovascular physiology concepts: Arterial baroreceptors. Downloaded from internet: URL cvphysiology.com/Blood %20Pressure/BP012.
Klucken et al., (2003). Neurochemical Research, 28(11), 1683-1691.
Koide et al., (1994). Gerontology, 40, 279-285.
Kono et al., (1990). Nippon Geka Gakkai Zasshi, 91, 1016-1022.
Kotzbauer et al., (2001). Journal of Molecular Neuroscience, 17(2), 225-232.
Kovacs et al., (2016). Acta Neuropathologica, 131, 911-923.
Kress et al., (2014). Annals of Neurology, 76, 845-861.
Laman and Weller, (2013). J. Neuroimmune Pharmacology, 8(4), 840-856.
Lee et al., (2006). Archives of Gerontology & Geriatrics, 42, 157-166.
Lee et al., (2015a). Journal of Neuroscience, 35(31), 11034-11044.
Lee et al., (1994). Microvascular Research, 48, 316-327.
Lee et al., (2015b). International Journal of Cardiovascular Imaging, 31(6), 1139-1148
Levine et al., (1982). Brain Research, 241, 31-41.
Levy et al., (1988). Radiology, 169(3), 773-778.
Louveau et al., (2016). Neuron, 91(5), 957-973
Louveau et al., (2015). Nature, 523, 337-341.
Maltz and Budinger, (2005). Physiological Measurement, 26(3), 293-307.
Mancini et al., (2014). PLoS One, 9(3): e92730.
Marengoni et al., (2011). Neurobiology of Aging, 32(7), 1336-1337.
Martin et al., (2012). American Journal of Physiology—Heart and Circulatory Physiology, 302, H1492-H1509.
Mavrocordatos et al., (2000). Journal of Neurosurgical Anesthesiology, 12(1), 10-14.
Mavroudis, (1978). Annals of Thoracic Surgery, 25, 259-271.
Mayer et al., (2011). Journal of Neurology, Neurosurgery, & Psychiatry. 82, 436-440.
McKee and Robinson, (2014). Alzheimers Dementia, 10(3 0), S242-S253.
Meyer et al., (2008). Proceedings of the National Academy of Sciences, 105(9), 3587-3592.
Mitchell et al., (2011). Brain, 134, 3398-3407.
Morovic et al., (2009). Journal of the Neurological Sciences, 283(1-2), 41-43.
Moussaud et al., (2014). Molecular Neurodegeneration, 9, 43. doi: 10.1186/1750-1326-9-43.
Muller et al., (2007). Atherosclerosis, 190, 143-149.
Muqtadar et al., (2012). Current Cardiology Reports, 14(6), 732-740.
Murkin et al., (1995). Journal of Thoracic and Cardiovascular Surgery, 110(2), 349-362.
Nakata et al., (1996). Artificial Organs, 20, 681-684.
Nation et al., (2012). Journal of Alzheimers Disease, 30(3), 595-603.
Nedelmann et al., (2009). Journal of Neurology, 256(6), 964-969.
Nedergaard, (2013). Science, 340, 1529-1530.
Neuropathology Group of the Medical Research Council Cognitive function and Ageing study. (2001). Lancet, 357, 169-75.
Ng et al., (2013). Clinical Epidemiology, 5, 135-145.
Nichols, (2005). American Journal Hypertension, 18(1), 3 S-10S.
Novak et al., (1998) Stroke, 29, 104-111.
O'Callaghan and Kenny, (2016). Yale Journal of Biological Medicine, 89(1), 59-71.
O'Connell, (1943). Brain, 66, 204-228.
O'Leary et al., (2011). Journal of Molecular Neuroscience, 45(3), 467-472.
Onoe et al., (1994). Journal of Thoracic and Cardiovascular Surgery, 108, 119-125.
O'Rourke et al., (2007). J. American College Cardiology, 50, 1-13.
Ott et al., (1997). Stroke, 28(2), 316-321.
Owler et al., (2004). Journal of Cerebral Blood Flow & Metabolism, 24(1), 17-23.
Padayachee et al., (1987). Annals of Thoracic Surgery, 44, 298-302.
Paini et al., (2007). Stroke, 38(1), 117-123.
Papa et al., (2003). American Journal of Emergency Medicine, 21(3), 250-251.
Papageorgiou et al., (2008). Clinical Neurology & Neurosurgery, 110(10), 1038-1040.
Park et al., (2007). Age & Ageing, 36, 157-163.
Pase et al., (2010). Journal of Hypertension, 28, 1724-1729.
Pase et al., (2013). Psychological Science. 24(11):2173-81
Pellicori et al., (2014). International Journal of Cardiology, 170(3), 364-370.
Perrin, (2007). Journal of the American Osteopathic Association, 107(6), 218-224.
Peters et al., (2009). Journal of Hypertension, 27(10), 2055-2062.
Plassman et al., (2007). Neuroepidemiology, 29(1-2), 125-132.
Poels et al., (2007). Stroke, 38, 888-892.
Preston et al., (2003). Neuropathology and Applied Neurobiology, 29, 106-117.
Pullicino et al., (2001). Journal of Stroke and Cerebrovascular Disease, 10, 178-182.
Purandare et al., (2006). British Medical Journal, 332(7550), 1119-1124.
Qiu et al., (2006). Archives of Internal Medicine, 166, 1003-1008.
Rams et al., (1993). Journal of Heart Valve Disease, 2, 37-41.
Rastas et al., (2007). Stroke, 38(5), 1454-1460.
Rawles and Rowland, (1986). Br Heart J, 56, 4-11.
Reitz et al., (2007). American Journal of Geriatric Cardiology, 16(3), 183-188.
Richardson et al., (1989). Lancet, 2, 941-944.
Roach et al., (1996). New England Journal of Medicine, 335(25), 1857-1863.
Robles Bayon et al., (2014). Neurologia, 29(2), 76-85.
Roberts et al., (2010). Neurobiology of Aging, 31, 1894-1902.
Rockwood et al., (1992). Journal of the American Geriatric Society, 40, 142-146.
Roher et al., (2012). Vascular Health and Risk Management, 8, 599-611.
Roher et al., (2011b). Alzheimers Dementia, 7(4), 445-455.
Roher et al., (2011a). Alzheimers Dementia, 7, 436-444.
Rosenfeld and Ford, (2010). Injury, 41(5), 437-443
Rubenstein, (1998). Lancet, 351(9098), 283-285.
Ruitenberg et al., (2005). Annals of Neurology, 57, 789-794.
Sanderson et al., (1972). Thorax, 27, 275-286.
Sauvé et al., (2009). Journal of Cardiac Failure, 15, 1-10.
Savageau et al., (1982). Journal of Thoracic & Cardiovascular Surgery, 84, 595-600.
Schley et al., (2006). Journal Theoretical Biology, 238, 962-974
Schreiber et al., (2003). Journal of Applied Physiology, 94, 1802-1805.
Schuff et al., (2009). Alzheimers Dementia, 5(6), 454-462.
Scuteri et al., (2005). Journal of Hypertension, 23, 1211-1216.
Scuteri et al., (2007). Journal of Hypertension, 25, 1035-1040.
Segal, (2000). Cellular and Molecular Neurobiology, 20, 183-196.
Selvarajah et al., (2009). European Radiology, 19, 1011-1018.
Sezai et al., (2005). Artificial Organs, 29, 708-713.
Sezai et al., (1999). Artificial Organs, 23, 280-285.
Sezai et al., (1996). Artificial Organs, 20, 139-142.
Sezai et al., (1997). Artificial Organs, 21, 830-835.
Shaw et al., (1986). Quarterly Journal of Medicine, 225, 59-68.
Shibata et al., (2000). The Journal of Clinical Investigation, 106, 1489-1499.
Shih et al., (2013). Nature Neuroscience, 16, 55-63.
Shima et al., (1998). Plastic Reconstrutive Surgery, 101, 33-41.
Silverberg et al., (2003). Lancet Neurology, 2, 506-511.
Silvestrini et al., (2011). J Alzheimer's Disease, 25(4), 719-726.
Simka, (2014). Journal of Vascular Diagnostics and Interventions, 2014(2), 1-13.
Simka et al., (2010). International Angiology, 29(2), 109-114.
Simon and Iliff, (2016). Biochimica et Biophysica Acta, 1862, 442-451.
Simmonds, (1952). Australian Journal of Experimental Biology and Medical Science, 30, 261-270.
Skoog, (1991). Alzheimer Disease & Associated Disorders, 13, 106-114.
Smith et al., (1986). Lancet, 1, 823-825.
Stefani et al., (2009). Journal of Neurological Science, 283, 109-115.
Stellos et al., (2012). Current Vascular Pharmacology, 12, 152-154.
Stemper and Pintar, (2014). Progress in Neurological Surgery, 28, 14-27.
Still, (1902). The Philosophy and Mechanical Principles of Osteopathy. Kansas City, Mo: Hudson-Kimberly Pub Co
Stivaros and Jackson, (2007). Neurotherapeutics, 4(3), 511-522.
Stopa et al., (2001). Experimental Neurology, 167, 40-47.
Sun et al., (2006). Proceedings of the National Academy of Science USA, 103, 18727-18732.
Tanaka et al., (1997). Neurosurgery, 40(6), 1161-1167.
Taylor, (1998). Annals of Thoracic Surgery, 65, 20-26.
Thoennissen et al., (2005). Journal of Thoracic and Cardiovascular Surgery, 130(4), 1159-1166.
Thurston, (1976). Microvasular Research, 11, 133-146.
Tohgi et al., (1998). Neuroradiology, 40(3), 131-137.
Toro et al., (2015). Current Neurovascular Research, 12,(4), 384-397.
Tranmer et al., (1986). Neurosurgery, 19, 724-731.
Tripathi, (1977). Experimental Eye Research, 25(Suppl), 65-116.
Trojano et al., (2003). Journal of Neurology, 250, 1456-1463.
Tzogias et al., (2011). International Angiology, 30(3), 212-220.
Uekermann et al., (2005). Journal of Heart Valve Disease, 14(3), 338-343.
Undar, (2005). American Society for Artificial Internal Organs Journal, 51, vi-x.
Undar et al., (2002a). Artificial Organs, 26, 919-923.
Undar et al., (2002b). American Society for Artificial Internal Organs Journal, 48, 90-95.
Valdueza et al., (2000). The Lancet, 355, 200-201.
Valecchi et al., (2010). Italian Journal of Anatomy and Embryology, 115(3), 185-189.
van Bruchem-Visser et al., (2009). Dementia and Geriatric Cognitive Disorders, 28, 320-324
van Oijen et al., (2007). Annals of Neurology, 61, 403-410.
Venugopal et al., (2004). 26th Annual International Conference of the IEEE, Engineering in Medicine and Biology Society, 3700-3703.
Verghese et al., (2003). Neurology, 61(12), 1667-1672.
Versluis et al., (2006). Journal of Biomechanics, 39, 339-347.
Vicenzini et al., (2007). European Neurology, 58, 84-89.
von der Weid and Zawieja, (2004). The International Journal of Biochemistry & Cell Biology, 36(7), 1147-1153.
Vranova et al., (2014). Journal of the Neurological Sciences, 343(1-2), 120-124.
Wahlin et al., (2014). Neurobiology of Aging, 35(2), 365-372.
Waldstein et al., (2008). Hypertension, 51, 99-104.
Wang and Olbricht, (2011). Journal Theoretical Biology, 274, 52-57.
Watson and Marx, (2002). Psychosomatics, 43, 413-417.
Weller et al., (2009). Acta Neuropathol, 117, 1-14.
Weller et al., (2010). Pathophysiology, 17, 295-306.
Weller et al., (1998). The American Journal of Pathology, 153, 725-733.
Wendell et al., (2012). Stroke, 43(12), 3319-3324.
Whedon and Glassey, (2009). Alternative Therapies in Health and Medicine, 15(3), 54-60.
Wolf, (2012). Archives of Neurology, 69, 567-571.
Wu et al. (2000). Anesthesiology, 93(2), 319-324.
Xie et al., (2013). Science, 342, 373-377.
Xiong et al., (2014). Journal of Neurotrauma, 31(16), 1396-1404.
Xu et al., (2016). Science Reports, 6, 31787. doi: 10.1038/srep31787.
Yamout et al., (2010). Multiple Sclerosis, 16, 1341-1348.
Yaneva-Sirakova et al., (2012). Journal of Cardiovascular Medicine (Hagerstown), 13(11), 735-740.
Zaharchuk et al., (2011). American Journal of Neuroradiology, 32, 1482-1489.
Zamboni et al., (2009a). Journal Neurological Neurosurgery Psychiatry. 80(4), 392-399.
Zamboni et al., (2009c) Journal of Neurological Sciences, 282(1-2): 21-27 Zamboni et al., (2009b). Functional Neurology, 24(3), 133-138.
Zanatta et al., (2012). Perfusion, 27(3), 199-206.
Zarow et al., (1997). Annual NY Academy of Science, 826, 147-160.
Zhang et al., (2015). Medicine, 94(47), 1-8.
Zhang et al., (2007) Journal of Biological Chemistry, 282, 10873-10880.
Zheng et al., (2014). PLos One, 9, e103451, doi: 10.1371/journal.pone. 0103451.
Zivadinov and Chung, (2013). BMC Medicine, 11, 260.
Zivadinov et al., (2011a). Neurology, 77, 138-144.
Zivadinov et al., (2011b). American Journal of Neuroradiology, 32, 938-946.
Zolla et al., (2015). Aging Cell, 14(4), 582-594.
Zuccala et al., (2001). Neurology, 57, 1986-92.

1. A biofluid flow assist device comprising:
(a) a collar configured to wrap at least partially around a subject's neck or limb;
(b) at least one biofluid flow assist mechanism configured to be position over a target when the collar is positioned on the subject's neck or limb;
(c) a controller operatively connected to the at least one biofluid flow assist mechanism; and
(d) a unit for providing power to the controller and the at least one biofluid assist mechanism.

2. The device of claim 1, wherein the biofluid is blood, lymph, and/or cerebrospinal fluid (CSF).

3. The device of claim 1, wherein the device comprises a first and second biofluid flow assist mechanisms.

4. The device of claim 1, wherein the biofluid flow assist mechanism is a mechanical, electrical, or hydraulic vascular assist mechanism.

5. The device of claim 4, wherein the biofluid flow assist mechanism targets a specific vessel, specific location, or a specific muscle(s) of the subject.

6. The device of claim 1, wherein the biofluid flow assist mechanism is configured to apply pressure directionally with the pressure having at least some lateral component along the fluid flow path.

7. The device of claim 1, further comprising at least one sensor.

8. The device of claim 7, wherein the sensor is an auditory sensor, pressure sensor, or a combination thereof.

9. The device of claim 7, further comprising a first sensor and a second sensor.

10. The device of claim 9, wherein the first sensor is positioned proximal to the biofluid flow assist mechanism relative to the subject's body and the second sensor is positioned distal to the biofluid flow assist mechanism relative to the subject's body or heart.

11. The device of claim 1, wherein the biofluid flow assist mechanism is a vascular assist mechanism.

12. The device of claim 11, wherein the vascular assist mechanism is a lymphatic vascular assist mechanism.

13. The device of claim 11, wherein the vascular assist mechanism is a venous vascular assist mechanism.

14. The device of claim 11, wherein the vascular assist mechanism is an arterial vascular assist mechanism.

15. The device of claim 1, further comprising both an arterial assist mechanism and a venous assist mechanism.

16. The device of claim 1, further comprising an arterial assist mechanism, a venous assist mechanism, and a lymphatic assist mechanism.

17. The device of claim 1, wherein the target is a specific vessel, specific location, a specific muscle.

18. The device of claim 1, wherein the target is the common carotid artery, internal jugular vein, external jugular vein, vertebral artery, vertebral vein, myodural bridge, or combinations thereof.

19. The device of claim 1, wherein the controller is programmed for one or more of biofluid flow assist onset, biofluid flow assist frequency, biofluid flow assist duration, biofluid flow assist force or biofluid flow assist stimulation pattern.

20. A method of assisting biofluid movement in a subjects brain, lymphatic system, or cardiovascular system comprising positioning the biofluid flow assist device of claim 1 on a subject, wherein the biofluid flow assist device is programmed to apply an appropriate series of pulses to a target for an appropriate duration.
